SEQUENCE LISTING (59 sequences)

SEQ ID NO: 1 - protein, 30 aa, synthetic construct
GAKGDKGSKGEVGFPGLAGSPGIPGSKGEQ

SEQ ID NO: 2 - protein, 300 aa, synthetic construct
GAKGDKGSKGEVGFPGLAGSPGIPGSKGEQGAKGDKGSKGEVGFPGLAGSPGIPGSKGEQGAKGDKGSKGEVGFPGLAGSPGIPGSKGEQGAKGDKGSKGEVGFPGLAGSPGIPGSKGEQGAKGDKGSKGEVGFPGLAGSPGIPGSKGEQGAKGDKGSKGEVGFPGLAGSPGIPGSKGEQGAKGDKGSKGEVGFPGLAGSPGIPGSKGEQGAKGDKGSKGEVGFPGLAGSPGIPGSKGEQGAKGDKGSKGEVGFPGLAGSPGIPGSKGEQGAKGDKGSKGEVGFPGLAGSPGIPGSKGEQ

SEQ ID NO: 3 - DNA, 900 bp, synthetic construct
  source 1..900: mol_type = other DNA
ggggctaaaggagacaagggcagcaagggcgaagtcggtttcccaggtctggctggtagcccgggcatcccgggttcaaagggtgaacaaggtgctaaaggcgacaaaggcagcaagggtgaggttggtttcccgggtctggcgggttctccaggcatcccgggtagcaaaggagaacaaggtgcgaaaggcgataaaggctccaagggtgaagtgggcttcccgggtttagccggtagcccaggtattccgggtagcaaaggcgaacagggtgcgaaaggcgacaaagggagtaagggcgaggtgggttttccgggtttggctggctcgccgggtattccgggttcaaagggcgaacagggcgcgaaaggtgataaaggcagcaaaggcgaggttggcttcccgggtctggcaggtagcccgggtatcccgggtagcaagggtgagcagggtgccaaaggcgacaaaggtagcaagggggaagtgggttttccgggactggcaggtagcccgggtatcccgggttctaagggcgagcagggtgcgaaaggtgacaaaggtagcaagggcgaggttggctttccgggcttggcgggtagcccgggcattccgggctccaagggtgaacaaggtgcgaaaggtgataaaggctctaagggtgaggttggttttccgggtctggcgggttccccgggcattccgggctcgaagggcgagcaaggtgctaaaggtgataagggctccaagggcgaggtgggtttcccgggcctggcaggctctccgggcatcccgggttcgaagggcgaacagggtgcgaaaggcgataaaggttccaagggcgaagtcggattccctggcctcgccggtagcccgggcatccctggctccaagggcgagcag

SEQ ID NO: 4 - protein, 39 aa, synthetic construct
GAKGDKGSKGEVGFPGLAGSPGIPGSKGEQGFMGPPGPQ

SEQ ID NO: 5 - protein, 312 aa, synthetic construct
GAKGDKGSKGEVGFPGLAGSPGIPGSKGEQGFMGPPGPQGAKGDKGSKGEVGFPGLAGSPGIPGSKGEQGFMGPPGPQGAKGDKGSKGEVGFPGLAGSPGIPGSKGEQGFMGPPGPQGAKGDKGSKGEVGFPGLAGSPGIPGSKGEQGFMGPPGPQGAKGDKGSKGEVGFPGLAGSPGIPGSKGEQGFMGPPGPQGAKGDKGSKGEVGFPGLAGSPGIPGSKGEQGFMGPPGPQGAKGDKGSKGEVGFPGLAGSPGIPGSKGEQGFMGPPGPQGAKGDKGSKGEVGFPGLAGSPGIPGSKGEQGFMGPPGPQ

SEQ ID NO: 6 - DNA, 936 bp, synthetic construct
  source 1..936: mol_type = other DNA
ggagctaaaggggacaaaggctccaaaggcgaagtcggtttcccgggcctggcgggtagcccgggtatcccgggtagcaagggtgagcaggggttcatgggtccaccgggcccacagggtgccaaaggtgataaaggttctaagggcgaggtgggtttcccggggctggcgggttctccgggcattccgggaagcaagggtgaacagggctttatgggtccgccaggtccgcagggtgcgaaaggtgataaaggcagcaagggagaagttggcttcccgggcctggcaggcagcccgggcattccggggtcgaagggcgaacaaggtttcatgggtccgcctggtccgcaaggtgcgaaaggtgataagggtagcaagggtgaagtgggttttccgggattagcgggttctccgggcattccgggttcaaaaggtgaacaaggctttatgggtccgcctggcccgcagggtgctaaaggcgacaagggtagcaaaggcgaggtaggtttcccgggtttggcgggcagcccgggcattccgggttccaagggcgagcagggttttatgggcccaccgggcccgcaaggcgcaaaaggtgataagggcagcaaaggcgaggtgggcttcccgggactggcaggttctccgggtatcccgggttccaagggtgagcagggtttcatgggcccaccgggtccgcagggtgcgaaaggcgacaaaggtagcaagggcgaagttggttttccgggcctggctggttcgccgggcatcccgggctccaagggcgagcaaggcttcatgggtccaccgggtccgcaaggtgccaaaggcgacaaaggtagcaagggcgaggttggttttccgggcttggctggtagccctggcatcccggggtccaagggtgaacagggctttatgggtccgccgggccctcaa

SEQ ID NO: 7 - protein, 51 aa, synthetic construct
GFPGFPGAKGDKGSKGEVGFPGLAGSPGIPGSKGEQGFMGPPGPQGQPGLP

SEQ ID NO: 8 - protein, 306 aa, synthetic construct
GFPGFPGAKGDKGSKGEVGFPGLAGSPGIPGSKGEQGFMGPPGPQGQPGLPGFPGFPGAKGDKGSKGEVGFPGLAGSPGIPGSKGEQGFMGPPGPQGQPGLPGFPGFPGAKGDKGSKGEVGFPGLAGSPGIPGSKGEQGFMGPPGPQGQPGLPGFPGFPGAKGDKGSKGEVGFPGLAGSPGIPGSKGEQGFMGPPGPQGQPGLPGFPGFPGAKGDKGSKGEVGFPGLAGSPGIPGSKGEQGFMGPPGPQGQPGLPGFPGFPGAKGDKGSKGEVGFPGLAGSPGIPGSKGEQGFMGPPGPQGQPGLP

SEQ ID NO: 9 - DNA, 918 bp, synthetic construct
  source 1..918: mol_type = other DNA
ggatttcccgggttcccgggtgccaaaggggataaaggttcaaagggcgaagtgggtttcccgggtttggctggtagcccgggtatcccgggtagcaaaggcgaacagggctttatgggtccgccaggaccgcagggtcaaccgggactgccgggttttccgggcttcccgggtgcgaaaggcgataaaggttccaagggtgaagttggttttccgggtcttgcaggcagcccgggtattccgggttccaagggtgaacagggtttcatgggtccaccgggcccacaaggtcagccgggtctgcctggtttcccgggcttcccgggtgccaaaggcgacaaaggtagcaagggcgaagttggctttccgggtctggcgggttcgccgggcattccgggctcgaagggcgagcagggtttcatgggcccaccgggtccgcagggtcagcctggcctgccgggattcccaggttttccgggagcgaaaggcgacaagggtagtaagggtgaggtcggttttccaggcttggcgggctctcccggtatcccgggctctaagggcgagcaaggctttatgggtccaccgggtccgcaaggtcaacctggattaccgggattcccaggctttccgggcgcgaaaggcgataaaggcagcaagggtgaggtgggcttcccgggcctcgcgggtagcccgggcatcccgggtagcaagggtgagcagggcttcatgggtcctccgggtccgcagggccaaccgggcctgccgggattcccgggtttcccgggcgctaaaggcgacaaaggcagcaagggtgaggttggttttccgggtctggcaggtagcccgggcattccgggctccaagggcgaacagggttttatgggtccaccgggccctcaaggtcagccgggcctgccg

SEQ ID NO: 10 - protein, 45 aa, synthetic construct
GFPGFPGAKGDKGSKGEVGFPGLAGSPGIPGSKGEQGFMGPPGPQ

SEQ ID NO: 11 - protein, 360 aa, synthetic construct
GFPGFPGAKGDKGSKGEVGFPGLAGSPGIPGSKGEQGFMGPPGPQGFPGFPGAKGDKGSKGEVGFPGLAGSPGIPGSKGEQGFMGPPGPQGFPGFPGAKGDKGSKGEVGFPGLAGSPGIPGSKGEQGFMGPPGPQGFPGFPGAKGDKGSKGEVGFPGLAGSPGIPGSKGEQGFMGPPGPQGFPGFPGAKGDKGSKGEVGFPGLAGSPGIPGSKGEQGFMGPPGPQGFPGFPGAKGDKGSKGEVGFPGLAGSPGIPGSKGEQGFMGPPGPQGFPGFPGAKGDKGSKGEVGFPGLAGSPGIPGSKGEQGFMGPPGPQGFPGFPGAKGDKGSKGEVGFPGLAGSPGIPGSKGEQGFMGPPGPQ

SEQ ID NO: 12 - DNA, 1080 bp, synthetic construct
  source 1..1080: mol_type = other DNA
ggatttcccgggtttccgggtgcgaaaggcgacaagggttccaagggtgaagttggttttccggggctcgctggtagcccgggcatcccgggtagcaagggcgaacagggtttcatgggcccaccgggcccgcagggttttccgggtttcccgggcgcgaaaggcgacaaaggcagcaagggtgaagtgggctttccgggcctggcgggtagtccgggcatcccgggctcgaagggcgaacagggctttatgggcccaccaggcccacagggcttcccgggattcccgggtgccaaaggcgataaaggttcaaagggcgaagtcggtttcccgggtctggcaggctctccgggcatcccgggctctaagggtgaacaaggtttcatgggtccaccgggcccgcagggctttccgggttttccgggtgcgaaaggcgacaagggcagcaaaggtgaggttggctttccgggcttggcgggtagcccgggtatcccgggtagcaagggtgagcagggtttcatgggtccgcctggaccgcaaggtttcccgggtttcccgggtgcgaaaggtgataaaggtagcaagggcgaggtaggttttccgggtctggcaggctccccgggcattccgggctctaagggtgagcagggtttcatgggtccgccaggtccgcaaggtttcccgggatttccgggtgctaaaggtgataaaggttccaaaggtgaagtgggttttccaggcctggccggcagcccgggcattccgggctccaagggcgagcagggcttcatgggtccgcctggccctcaaggcttccccggcttcccgggagctaagggcgacaaaggttccaagggcgaggtgggcttcccgggcttggcgggtagcccgggcattccgggtagcaagggtgagcaaggctttatgggtccgccaggtccgcaagggttcccgggattcccgggtgcgaaaggtgataaaggctcgaagggtgaggttggttttccgggtctggcaggtagcccggggattccgggtagcaaaggcgaacaaggtttcatgggtcctccgggtccacag

SEQ ID NO: 13 - protein, 39 aa, synthetic construct
GFPGFPGAKGDKGSKGEVGFPGLAGSPGIPGSKGEQGFM

SEQ ID NO: 14 - protein, 312 aa, synthetic construct
GFPGFPGAKGDKGSKGEVGFPGLAGSPGIPGSKGEQGFMGFPGFPGAKGDKGSKGEVGFPGLAGSPGIPGSKGEQGFMGFPGFPGAKGDKGSKGEVGFPGLAGSPGIPGSKGEQGFMGFPGFPGAKGDKGSKGEVGFPGLAGSPGIPGSKGEQGFMGFPGFPGAKGDKGSKGEVGFPGLAGSPGIPGSKGEQGFMGFPGFPGAKGDKGSKGEVGFPGLAGSPGIPGSKGEQGFMGFPGFPGAKGDKGSKGEVGFPGLAGSPGIPGSKGEQGFMGFPGFPGAKGDKGSKGEVGFPGLAGSPGIPGSKGEQGFM

SEQ ID NO: 15 - DNA, 936 bp, synthetic construct
  source 1..936: mol_type = other DNA
ggatttcccgggttcccgggtgcgaaaggtgataaaggcagcaagggtgaagtcggttttccgggtctggcaggcagcccgggtatcccgggtagcaaaggcgaacagggctttatgggtttcccgggcttcccaggtgcgaagggcgataaaggttcgaaaggtgaggtaggtttcccgggtttagcaggttccccgggcattccgggcagcaagggtgaacagggtttcatgggctttccgggcttcccaggagctaaaggcgacaaaggttctaagggtgaagtgggcttcccgggtctggctggtagcccgggcatcccgggctccaagggtgagcagggtttcatgggttttccgggcttcccaggcgcgaaaggcgacaaaggcagcaagggcgaggtgggttttccgggtttggcgggtagcccgggtattccgggttcgaagggtgaacaaggtttcatgggttttccgggattcccaggcgcgaaaggcgataagggcagcaagggcgaggttggcttcccgggactggccggaagcccgggtatcccgggatctaagggcgaacaaggctttatgggtttcccgggttttcctggtgcgaaaggcgataaaggctccaagggcgaggttggttttccaggcctggctggctctccgggcattccgggtagtaagggtgagcagggttttatgggttttccgggcttcccgggtgcaaagggtgacaaaggtagcaagggtgaagttggctttccgggtctggcgggttccccgggcattccgggtagcaaaggtgagcaaggttttatgggttttccgggcttcccgggtgccaaaggcgacaaaggtagcaagggagaggtgggcttcccgggattggcgggttccccgggcatcccgggctcaaagggtgaacagggtttcatg

SEQ ID NO: 16 - protein, 36 aa, synthetic construct
GFPGFPGAKGDKGSKGEVGFPGLAGSPGIPGSKGEQ

SEQ ID NO: 17 - protein, 360 aa, synthetic construct
GFPGFPGAKGDKGSKGEVGFPGLAGSPGIPGSKGEQGFPGFPGAKGDKGSKGEVGFPGLAGSPGIPGSKGEQGFPGFPGAKGDKGSKGEVGFPGLAGSPGIPGSKGEQGFPGFPGAKGDKGSKGEVGFPGLAGSPGIPGSKGEQGFPGFPGAKGDKGSKGEVGFPGLAGSPGIPGSKGEQGFPGFPGAKGDKGSKGEVGFPGLAGSPGIPGSKGEQGFPGFPGAKGDKGSKGEVGFPGLAGSPGIPGSKGEQGFPGFPGAKGDKGSKGEVGFPGLAGSPGIPGSKGEQGFPGFPGAKGDKGSKGEVGFPGLAGSPGIPGSKGEQGFPGFPGAKGDKGSKGEVGFPGLAGSPGIPGSKGEQ

SEQ ID NO: 18 - DNA, 1080 bp, synthetic construct
  source 1..1080: mol_type = other DNA
ggatttcccgggttcccaggtgccaagggtgataaaggcagcaagggcgaggtgggcttcccgggcctggctggttcaccgggcattccgggcagtaagggtgaacagggttttccgggtttccccggcgctaaaggcgacaagggtagcaagggggaagtaggcttcccgggtttagctggctctccgggcattccgggctccaagggcgaacagggtttcccgggctttccgggagcgaaaggcgacaaaggcagcaagggcgaagttggtttcccgggtctggcaggttctccgggtatcccgggtagcaaaggcgagcagggtttccccggcttcccgggtgcgaaaggtgataaaggtagcaagggtgaggtgggtttccctggcctggccggttctccgggcatcccgggttctaagggagaacaaggttttccgggttttccgggtgcgaaaggcgacaaagggtccaagggtgaggttggttttccgggtctggcgggttcaccgggcatcccgggtagcaagggcgaacaaggctttccaggcttcccgggtgcgaaaggtgacaaaggttccaagggcgaagttggttttccgggtttggcgggtagcccgggtatcccgggttccaagggtgagcaaggttttccgggattcccgggtgccaaaggtgacaaaggttcgaagggtgaagtgggttttccaggcttggcgggtagcccgggtatcccgggttccaagggtgagcagggcttcccgggtttcccgggtgcaaaaggtgataaaggctccaaaggtgaggtgggcttccctggtctcgcgggtagcccgggcatcccgggtagcaaaggtgagcagggtttcccgggctttccaggagctaaaggggataaaggcagcaagggcgaggttggcttcccgggtctggcgggttcgccgggcattccgggcagcaaaggcgaacagggctttccgggttttccgggtgcaaagggtgataaaggtagcaagggcgaggtcggttttccgggcctggcaggtagcccaggcattccgggttcgaagggcgaacaa

SEQ ID NO: 19 - protein, 45 aa, synthetic construct
GAKGDKGSKGEVGFPGLAGSPGIPGSKGEQGFMGPPGPQGQPGLP

SEQ ID NO: 20 - protein, 360 aa, synthetic construct
GAKGDKGSKGEVGFPGLAGSPGIPGSKGEQGFMGPPGPQGQPGLPGAKGDKGSKGEVGFPGLAGSPGIPGSKGEQGFMGPPGPQGQPGLPGAKGDKGSKGEVGFPGLAGSPGIPGSKGEQGFMGPPGPQGQPGLPGAKGDKGSKGEVGFPGLAGSPGIPGSKGEQGFMGPPGPQGQPGLPGAKGDKGSKGEVGFPGLAGSPGIPGSKGEQGFMGPPGPQGQPGLPGAKGDKGSKGEVGFPGLAGSPGIPGSKGEQGFMGPPGPQGQPGLPGAKGDKGSKGEVGFPGLAGSPGIPGSKGEQGFMGPPGPQGQPGLPGAKGDKGSKGEVGFPGLAGSPGIPGSKGEQGFMGPPGPQGQPGLP

SEQ ID NO: 21 - DNA, 1080 bp, synthetic construct
  source 1..1080: mol_type = other DNA
ggggctaaaggagacaagggcagcaagggcgaggttggtttcccgggcttggcgggttctccgggtatcccgggctccaagggtgaacagggttttatgggtccgccaggtccgcagggccaacctggtctgccgggcgcgaaaggtgacaaaggtagcaagggcgaagttggttttccgggcctggccggcagcccgggcattccgggctccaaaggcgagcaaggcttcatgggtccgcctggcccacagggtcaaccgggtctgccgggcgcaaaaggcgataaaggcagcaagggcgaagtaggtttcccgggccttgctggctctccgggtattccgggtagcaagggtgaacagggttttatgggcccaccgggaccacagggtcaaccgggtctgccgggtgctaaaggtgataaaggcagcaagggcgaggttggttttccgggattggcgggttctccgggcatcccgggttccaagggcgaacaaggtttcatgggaccgcctggcccacagggtcaaccgggtctgcctggtgcaaaaggcgacaaaggtagcaaaggcgaggtcggttttccgggcctcgcgggtagcccgggaatcccgggtagtaagggcgaacagggtttcatgggtccaccgggcccacagggtcagccgggtttaccgggtgccaaaggcgataaagggtcgaagggcgaggtgggcttcccgggtctggcaggcagcccgggtattccgggctccaagggtgagcagggttttatgggtccaccgggaccgcaaggtcaaccgggtctgccgggcgctaaaggcgataagggtagcaaaggcgaggtgggcttcccgggtctggcgggtagcccgggcattccgggttccaagggtgagcagggtttcatgggtccgcctggtcctcaaggccagccggggctgcccggcgcgaaaggtgacaaaggtagcaagggcgaagtgggttttccgggtttggcgggttcaccgggcatcccgggctcgaagggtgaacagggtttcatgggtcctccgggcccgcagggacaaccgggactgccg

SEQ ID NO: 22 - protein, 33 aa, synthetic construct
GAKGDKGSKGEVGFPGLAGSPGIPGSKGEQGFM

SEQ ID NO: 23 - protein, 330 aa, synthetic construct
GAKGDKGSKGEVGFPGLAGSPGIPGSKGEQGFMGAKGDKGSKGEVGFPGLAGSPGIPGSKGEQGFMGAKGDKGSKGEVGFPGLAGSPGIPGSKGEQGFMGAKGDKGSKGEVGFPGLAGSPGIPGSKGEQGFMGAKGDKGSKGEVGFPGLAGSPGIPGSKGEQGFMGAKGDKGSKGEVGFPGLAGSPGIPGSKGEQGFMGAKGDKGSKGEVGFPGLAGSPGIPGSKGEQGFMGAKGDKGSKGEVGFPGLAGSPGIPGSKGEQGFMGAKGDKGSKGEVGFPGLAGSPGIPGSKGEQGFMGAKGDKGSKGEVGFPGLAGSPGIPGSKGEQGFM

SEQ ID NO: 24 - DNA, 990 bp, synthetic construct
  source 1..990: mol_type = other DNA
ggggctaaaggagacaaaggttcgaaaggcgaggtgggcttcccaggtctggccggttccccgggcattccgggtagcaaaggcgaacaaggtttcatgggtgctaaaggcgataaaggtagcaagggtgaggttggcttcccaggcctggctggttcgccgggcattccgggctctaagggtgaacaaggtttcatgggtgcaaaaggtgataagggtagcaagggagaagtcggttttccgggattggcgggtagcccgggtatcccgggcagcaagggcgagcagggttttatgggtgcaaagggcgacaaaggtagcaagggtgaggtgggctttccgggcctcgcgggtagccctggcatcccgggttccaaaggtgagcaaggcttcatgggtgctaaaggtgataaaggctccaaaggtgaagtgggttttccgggcctggcgggtagcccgggcattccgggaagcaagggcgaacagggttttatgggcgcgaagggtgataaaggtagtaagggcgaagttggtttcccgggcctggctggctctccgggtatcccgggctccaaaggcgagcagggtttcatgggtgcgaaaggtgacaagggtagcaagggtgaggtgggtttcccaggtttggcgggtagcccgggcattccgggtagcaagggtgaacaaggtttcatgggtgcgaaaggtgacaaaggcagcaagggcgaggttggtttcccgggtctggcgggtagcccgggcatcccgggctctaagggcgagcagggttttatgggtgccaaaggcgacaagggctcaaagggtgaagtcggttttccgggtttagccggttccccgggcatcccgggttctaagggtgaacagggcttcatgggcgcgaaaggagataaaggcagcaaaggggaagttggttttccaggcctggcaggctcgccgggtatcccgggttccaagggcgagcagggttttatg

SEQ ID NO: 25 - protein, 36 aa, synthetic construct
GLPGTPGPTGPAGQKGEPGSDGIPGSAGEKGEPGLP

SEQ ID NO: 26 - protein, 360 aa, synthetic construct
GLPGTPGPTGPAGQKGEPGSDGIPGSAGEKGEPGLPGLPGTPGPTGPAGQKGEPGSDGIPGSAGEKGEPGLPGLPGTPGPTGPAGQKGEPGSDGIPGSAGEKGEPGLPGLPGTPGPTGPAGQKGEPGSDGIPGSAGEKGEPGLPGLPGTPGPTGPAGQKGEPGSDGIPGSAGEKGEPGLPGLPGTPGPTGPAGQKGEPGSDGIPGSAGEKGEPGLPGLPGTPGPTGPAGQKGEPGSDGIPGSAGEKGEPGLPGLPGTPGPTGPAGQKGEPGSDGIPGSAGEKGEPGLPGLPGTPGPTGPAGQKGEPGSDGIPGSAGEKGEPGLPGLPGTPGPTGPAGQKGEPGSDGIPGSAGEKGEPGLP

SEQ ID NO: 27 - DNA, 1080 bp, synthetic construct
  source 1..1080: mol_type = other DNA
ggactacccgggactccgggcccaaccggcccagcaggccaaaagggtgaaccggggtccgacggcattccgggcagcgcaggtgagaaaggcgaaccgggcttgccggggttgccgggaaccccgggtccgaccggtccagctggccagaaaggtgagccaggaagcgatggcatcccgggctcggccggtgaaaaaggcgagccgggtctcccgggtctgccgggaaccccgggtccgacaggcccggctggtcagaagggcgagccgggtagcgacggcatcccgggcagcgctggtgaaaaaggtgagcccggtctgccgggcttgcccggcaccccgggaccgacgggcccagcaggccagaagggcgaaccgggttcggatggtattccgggctctgccggtgagaaaggcgagcccggcttgccaggcctgcctggcaccccgggtccgaccggtccggcgggccaaaaaggcgagccgggtagcgatggtatcccgggctcagccggtgagaagggtgagccgggcctgccgggcctgcctggtacgccgggtccgaccggtccggcgggccaaaagggtgaaccgggctccgacggcattccgggttctgcgggtgagaaaggtgaaccgggcctgccgggcctgcccggcaccccgggtcctacgggtccggctggtcagaaaggcgagccgggctccgatggcattccgggttctgcgggtgagaagggtgaaccgggcttgccaggtctgccgggcaccccgggtccgacgggtccggcgggtcagaagggtgagccgggttccgatggtatcccgggcagcgcgggagaaaaaggtgaaccgggtctgccgggtcttccgggtactccgggtccgaccggccctgcgggtcagaagggtgagccgggaagcgacggcatcccgggcagcgcgggggagaagggtgaaccgggtttacctggcctgccgggaaccccgggccctaccggtccggcgggtcaaaagggcgaaccgggcagcgacggtatcccgggtagcgcaggcgaaaaaggtgaaccgggcctgccg

SEQ ID NO: 28 - protein, 30 aa, synthetic construct
GPTGPAGQKGEPGSDGIPGSAGEKGEPGLP

SEQ ID NO: 29 - protein, 300 aa, synthetic construct
GPTGPAGQKGEPGSDGIPGSAGEKGEPGLPGPTGPAGQKGEPGSDGIPGSAGEKGEPGLPGPTGPAGQKGEPGSDGIPGSAGEKGEPGLPGPTGPAGQKGEPGSDGIPGSAGEKGEPGLPGPTGPAGQKGEPGSDGIPGSAGEKGEPGLPGPTGPAGQKGEPGSDGIPGSAGEKGEPGLPGPTGPAGQKGEPGSDGIPGSAGEKGEPGLPGPTGPAGQKGEPGSDGIPGSAGEKGEPGLPGPTGPAGQKGEPGSDGIPGSAGEKGEPGLPGPTGPAGQKGEPGSDGIPGSAGEKGEPGLP

SEQ ID NO: 30 - DNA, 900 bp, synthetic construct
  source 1..900: mol_type = other DNA
ggtcccacaggaccggcaggccagaaaggtgagccgggttccgacggcatcccgggttcggcgggtgagaaaggcgagccgggtttaccgggtccgaccggtcccgcgggtcaaaagggcgagccgggtagcgatggcattccgggttctgcgggtgaaaagggcgaaccgggcctcccgggtcctaccggtccggcgggtcagaaaggcgaaccgggcagcgatggcatcccgggcagcgcgggcgagaaaggcgaaccgggcctgccgggcccgaccggaccagctgggcaaaaaggtgaaccgggcagcgacggcatcccgggttctgcaggcgagaaaggtgaaccaggcctgccgggaccgaccggtccggcaggccagaaaggtgagcctggcagtgatggtattccgggttctgccggtgaaaaaggtgagccgggcctgccggggccaacgggcccagccggacaaaaaggtgagccgggttccgacggcatcccgggctccgccggtgaaaagggtgagccgggcctgcctggcccaacgggtccggctggccaaaagggcgagccgggtagcgacggcattccgggcagcgcgggtgagaagggtgagccgggattgccgggtccgactggtcctgcgggccagaagggtgaaccgggttccgacggcatccccggctcggcgggtgaaaagggcgaaccgggtctgcctggtccgaccggcccagcgggtcagaagggtgaaccgggtagcgatggaatcccgggtagcgctggtgaaaagggcgagccgggcctgccgggtccgaccggtccggcaggccagaagggtgaaccgggtagcgatggtattccgggtagcgcgggcgaaaaaggtgagccgggcttgccg

SEQ ID NO: 31 - protein, 33 aa, synthetic construct
GFPGFPGAKGDKGSKGEVGFPGLAGSPGIPGSK

SEQ ID NO: 32 - protein, 330 aa, synthetic construct
GFPGFPGAKGDKGSKGEVGFPGLAGSPGIPGSKGFPGFPGAKGDKGSKGEVGFPGLAGSPGIPGSKGFPGFPGAKGDKGSKGEVGFPGLAGSPGIPGSKGFPGFPGAKGDKGSKGEVGFPGLAGSPGIPGSKGFPGFPGAKGDKGSKGEVGFPGLAGSPGIPGSKGFPGFPGAKGDKGSKGEVGFPGLAGSPGIPGSKGFPGFPGAKGDKGSKGEVGFPGLAGSPGIPGSKGFPGFPGAKGDKGSKGEVGFPGLAGSPGIPGSKGFPGFPGAKGDKGSKGEVGFPGLAGSPGIPGSKGFPGFPGAKGDKGSKGEVGFPGLAGSPGIPGSK

SEQ ID NO: 33 - DNA, 990 bp, synthetic construct
  source 1..990: mol_type = other DNA
ggatttcccgggttcccaggcgcaaaaggtgataaaggcagcaagggcgaggttggttttccaggtttagctggtagcccgggtatcccgggtagcaagggcttcccgggttttccgggtgctaaaggcgacaaaggctccaagggcgaagtcggtttcccgggtttggcgggtagcccgggtatcccgggtagtaagggctttccgggattcccaggcgcgaaaggtgacaaaggtagcaagggcgaagttggcttcccgggtttggcgggttccccgggtatcccggggtccaagggcttccccggattcccgggcgcgaaaggcgataaaggtagcaagggtgaagtgggttttccgggtctcgctggcagcccgggtattccgggctccaagggctttccaggctttccgggtgcgaaaggcgataaaggtagcaagggtgaggtgggttttccgggtctggcaggtagccctggcatcccgggctcgaaggggttcccgggcttcccgggagccaagggtgataaaggttctaagggtgaggtcggttttccgggcctggccggtagccctggtatcccggggagcaagggtttcccgggttttccgggtgccaaaggcgataaaggctctaagggcgaggtgggcttccccggtctggcgggtagcccgggtattccgggttctaagggcttcccgggttttccgggtgcgaaaggtgacaagggctccaagggtgaagttggttttccgggtctggctggtagcccgggtatcccgggtagcaagggcttcccgggtttcccgggcgcgaaaggcgacaaaggttcaaagggtgaagttggttttcctggcctggcaggcagcccgggcattccgggttccaaaggttttccgggcttcccgggtgcgaaaggtgacaaaggctcgaagggtgaggtgggcttcccgggtctggcaggttctcctggcattccgggttcgaaa

SEQ ID NO: 34 - protein, 6 aa, synthetic construct
GFPGFP

SEQ ID NO: 35 - protein, 15 aa, synthetic construct
GFMGPPGPQGQPGLP

SEQ ID NO: 36 - protein, 6 aa, synthetic construct
GLPGTP

SEQ ID NO: 37 - protein, 6 aa, synthetic construct
ENLYFQ

SEQ ID NO: 38 - DNA, 18 bp, synthetic construct
  source 1..18: mol_type = other DNA
gaaaacctgtatttccag

SEQ ID NO: 39 - protein, 36 aa, synthetic construct
GSKGEVGFPGLAGSPGIPGSKGEQGFMGPPGPQGAK

SEQ ID NO: 40 - protein, 33 aa, synthetic construct
GEVGFPGLAGSPGIPGSKGEQGFMGPPGPQGAK

SEQ ID NO: 41 - protein, 36 aa, synthetic construct
GEVGFPGLAGSPGIPGSKGEQGFMGPPGPQGARGDK

SEQ ID NO: 42 - protein, 15 aa, synthetic construct
GEQGFMGPPGPQGAK

SEQ ID NO: 43 - protein, 18 aa, synthetic construct
GEQGFMGPPGPQGAKGDK

SEQ ID NO: 44 - protein, 24 aa, synthetic construct
GDKGSKGEVGFPGLAGSPGIPGSK

SEQ ID NO: 45 - protein, 21 aa, synthetic construct
GSKGEVGFPGLAGSPGIPGSK

SEQ ID NO: 46 - protein, 33 aa, synthetic construct
GSKGEVGFPGLAGSPGIPGSKGEQGFMGPPGPQ

SEQ ID NO: 47 - protein, 18 aa, synthetic construct
GEVGFPGLAGSPGIPGSK

SEQ ID NO: 48 - protein, 30 aa, synthetic construct
GEVGFPGLAGSPGIPGSKGEQGFMGPPGPQ

SEQ ID NO: 49 - protein, 12 aa, synthetic construct
GEQGFMGPPGPQ

SEQ ID NO: 50 - protein, 27 aa, synthetic construct
GSKGEVGFPGLAGSPGIPGSKGEQGAK

SEQ ID NO: 51 - protein, 24 aa, synthetic construct
GEVGFPGLAGSPGIPGSKGEQGAK

SEQ ID NO: 52 - protein, 27 aa, synthetic construct
GEVGFPGLAGSPGIPGSKGEQGAKGDK

SEQ ID NO: 53 - protein, 21 aa, synthetic construct
GSKGEVGFPGLAGSPGIPGSK

SEQ ID NO: 54 - protein, 18 aa, synthetic construct
GEVGFPGLAGSPGIPGSK

SEQ ID NO: 55 - protein, 21 aa, synthetic construct
GEVGFPGLAGSPGIPGSKGEQ

SEQ ID NO: 56 - protein, 6 aa, synthetic construct
ENLYFQ

SEQ ID NO: 57 - DNA, 18 bp, synthetic construct
  source 1..18: mol_type = other DNA
gaaaacctgtatttccag

SEQ ID NO: 58 - protein, 5 aa, synthetic construct
FPGFP

SEQ ID NO: 59 - protein, 4 aa, synthetic construct
PGFP

(19) United States
(12) Patent Application Publication
(10) Pub. No.: US 2025/0263465 A1
(43) Pub. Date: Aug. 21, 2025

(54) METHOD FOR PREPARING BIOSYNTHETIC HUMAN STRUCTURAL MATERIAL TYPE IV COLLAGEN

(71) Applicant: Shanxi Jinbo Bio-Pharmaceutical Co. LTD., Taiyuan (CN)

(72) Inventors: Xia Yang, Taiyuan (CN); Zengyao Liu, Taiyuan (CN); Xiaobin Lan, Taiyuan (CN); Lingling Wang, Taiyuan (CN); Yongjian Zhang, Taiyuan (CN); Xin Liu, Taiyuan (CN); Xiaobo Zhang, Taiyuan (CN); Zhenrui He, Taiyuan (CN)

(21) Appl. No.: 19/203,486

(22) Filed: May 9, 2025

Related U.S. Application Data
(63) Continuation of application No. PCT/CN2023/140632, filed on Dec. 21, 2023.

(30) Foreign Application Priority Data
Oct. 25, 2023 (CN) 202311391711.1

(51) Int. Cl. C07K 14/78; A61K 8/65; C12N 15/70

(52) U.S. Cl. CPC C07K 14/78; A61K 8/65; C12N 15/70

(57) ABSTRACT

Provided herein is a method of biosynthesizing human body structural material type IV collagen. Provided herein is a recombinant collagen including the sequence represented by SEQ ID NO: 1, etc. The recombinant collagen of the present application exhibits excellent cell-adhesion activity. The method of the present application utilizes genetic engineering techniques to produce the recombinant collagen, thereby overcoming the drawbacks of the prior art.

### CROSS-REFERENCE TO RELATED APPLICATIONS

This application is a continuation of International Patent Application No. PCT/CN 2023/140632 filed on Dec. 21, 2023 which claims the priority benefit of the Chinese Patent Application No. 202311391711.1, filed on Oct. 25, 2023, titled “A Method for Preparing Biosynthetic Human Structural Material Type IV Collagen”. The content of the aforementioned application is incorporated herein by reference.

### SEQUENCE LISTING

A sequence listing contained in the file named LS-SHANXI-7001US_SL which is 67,042 bytes and created on May 6, 2025, is filed electronically herewith and incorporated by reference in its entirety.

### Technical Field

The present disclosure pertains to the field of biomedicine and relates to a recombinant type IV humanized collagen, as well as its preparation method and use.

### Background of the Disclosure

Collagen (abbreviated as COL) is a helical fibrous functional protein constituted by three peptide chains. As the principal component of the extracellular matrix, it is abundant in quantity and widely distributed. In the human body, collagen accounts for 25%-30% of the total protein content and is mainly present in the skin, tendons, and bones. It plays a crucial role in safeguarding and connecting various tissues, exerting significant physiological functions in vivo. Type IV collagen is a key protein that, together with laminin, constitutes the basement membrane. Its structural unit is a trimer with a helical structure composed of three a chains. While the subunits of the trimer vary depending on the different tissue, in numerous tissues such as the liver, the trimer consists of two α1 chains and one α2 chain. The type IV collagen molecule features unique structures at both ends of the TH domain that forms the helical structure. The N-terminal structure is designated as the 7S domain, and the C-terminal structure is termed the NC1 domain.

In recent years, collagen has gained significant attention as a research hotspot due to its favorable biocompatibility, degradability, low antigenicity and unique biological structure. It finds extensive applications in multiple fields such as biomedicine, cosmetics, health products, and food.

There are 28 different types of collagen found in the human body, which can be classified into two major categories: fibrous collagen and non-fibrous collagen, depending on whether their structure is fibrous. Fibrous collagen mainly undertakes the function of a cell scaffold, fixing cell positions and acting as anchors, while also providing tensile strength and stiffness to tissues. Non-fibrous collagen, on the other hand, is further subdivided into basal collagen, short-chain collagen, transmembrane collagen, etc., each fulfilling distinct functions.

The traditional method for producing collagen involves purifying animal-derived tissues through acid-hydrolysis, alkali-hydrolysis, or enzymatic-hydrolysis to extract collagen derivatives. However, the collagen obtained by these methods often loses its original biological activity, exhibits poor water solubility, has difficulty in binding to the human body, confronts challenges in averting the risks of viral infection and sensitization, and is unable to fully perform its true function. Some research institutions have attempted to express human collagen in vitro using conventional recombinant expression methods, but these approaches are costly, time-consuming, and not amenable to large-scale production.

Therefore, there is an urgent demand in the market for a collagen material with excellent biomaterial properties, a high degree of amino-acid sequence homology with the human body, and the capacity for large-scale preparation within an industrial system.

### SUMMARY OF THE INVENTION

The inventors carried out a large-scale screening of the functional region of human type IV collagen and identified 11 recombinant collagens. These recombinant collagens can be expressed in Escherichia coli and subsequently purified. Furthermore, the inventors found that these recombinant collagens exhibit higher yields, a higher purity of the collagens after fine purification, and higher activity compared to the positive control (bovine type I collagen) in cell adhesion activity assays.

In one aspect, the present disclosure provides a recombinant collagen comprising one or more repeating units linked directly or via a linker, wherein the repeating unit comprises an amino acid sequence selected from the group consisting of SEQ ID NO: 1 (Gakgdkgskgevgfpglagspgipgskgeq) or 28 (Gptgpagqkgepgsdgipgsagekgepglp), or a variant thereof, and wherein the variant is (1) an amino acid sequence having one or more amino acid residue mutations in said amino acid sequence or (2) an amino acid sequence having at least 80%, 85%, 86%, 87%, 88%, 89%, 90%, 91%, 92%, 93%, 94%, 95%, 96%, 97%, 98%, 99% or 100% identity to said amino acid sequence.

In one embodiment, the number of repeating units is 2-50 repeating units, such as 2-45, 2-40, 2-35, 2-30, 2-25, 2-20, 2-15, 4-10 or 6-10 repeating units. For example, the number of the repeating units may be 2, 3, 4, 5, 6, 7, 8, 9, 10, 12, 14, 16, 18, 20, 30, 40, or 50, or range therebetween.

In one embodiment, the linker comprises one or more amino acid residues, such as 1-10, 1-9, 1-8, 1-7, 1-6, 1-5, 1-4, 1-3 or 1-2 amino acid residues.

In one embodiment, the mutation is selected from the group consisting of substitution, addition, insertion or deletion.

In one embodiment, the substitution is a conservative amino acid substitution.

In one embodiment, the recombinant collagen is a recombinant human type IV collagen or a recombinant type IV humanized collagen.

In one embodiment, the recombinant collagen has cell adhesion activity.

In one embodiment, the variant of SEQ ID NO: 1 comprises the following mutations: the addition of Gfpgfp (SEQ ID NO: 34) or the N-terminal truncated fragment of SEQ ID NO: 34 with a length of 1-5 amino acid residues to the N-terminus of the amino acid sequence of SEQ ID NO: 1, and/or the addition of GFM GPPGPQGQPGLP (SEQ ID NO: 35) or the C-terminal truncated fragment of SEQ ID NO: 35 with a length of 1-14 amino acid residues to the C-terminus of the amino acid sequence of SEQ ID NO: 1, or the truncation of the amino acid sequence of SEQ ID NO: 1 at C-terminus by 1-5 amino acid residues.

In one embodiment, the variant of SEQ ID NO: 28 comprises the following mutations: the addition of Glpgtp (SEQ ID NO: 36) or the N-terminal truncated fragment thereof with a length of 1-5 amino acid residues to the N-terminus of the amino acid sequence of SEQ ID NO: 28.

In one embodiment, the variant of SEQ ID NO: 1 is selected from the group consisting of SEQ ID NO: 4 (Gakgdkgskgevgfpglagspgipgskgeqgfmgppgpq), 7 (GFPGFPGAKGDK GSK GEVGFPGLAGSPGIPGSK GEQGFM GPPGPQGQPGLP), 10 (Gfpgfpgakgdkgskgevgfpglagspgipgskgeqgfmgppgpq), 13 (Gfpgfpgakgdkgskgevgfpglagspgipgskgeqgfm), 16 (Gfpgfpgakgdkgskgevgfpglagspgipgskgeq), 19 (Gakgdkgskgevgfpglagspgipgskgeqgfmgppgpqgqpglp), 22 (Gakgdkgskgevgfpglagspgipgskgeqgfm), or 31 (Gfpgfpgakgdkgskgevgfpglagspgipgsk).

In one embodiment, the variant of SEQ ID NO: 28 is SEQ ID NO: 25 (Glpgtpgptgpagqkgepgsdgipgsagekgepglp).

In one embodiment, the recombinant collagen comprises an amino acid sequence selected from the group consisting of SEQ ID NOs: 2, 5, 8, 11, 14, 17, 20, 23, 26, 29 and 32, or a variant thereof, wherein the variant is (1) an amino acid sequence having one or more amino acid residue mutations in said amino acid sequence or (2) an amino acid sequence having at least 80%, 85%, 86%, 87%, 88%, 89%, 90%, 91%, 92%, 93%, 94%, 95%, 96%, 97%, 98%, 99% or 100% identity to said amino acid sequence.

In one embodiment, the mutation is selected from the group consisting of a substitution, an addition, an insertion or a deletion. In one embodiment, the substitution is a conservative amino acid substitution.

In another aspect, provided is a nucleic acid encoding a recombinant collagen described herein. In one embodiment, the nucleic acid comprises a codon-optimized nucleotide sequence. In one embodiment, the nucleotide sequence is a nucleotide sequence codon-optimized for expression in eukaryotic host cells or prokaryotic host cells, such as yeast or E. coli. In one embodiment, the nucleic acid comprises a nucleotide sequence selected from the group consisting of SEQ ID NO: 3, 6, 9, 12, 15, 18, 21, 24, 27, 30 or 33.

In another aspect, provided is a vector comprising a nucleic acid described herein. In one embodiment, the vector comprises an expression control element, nucleotides of a purification tag and/or nucleotides of a leader sequence operably linked to the nucleic acid. In one embodiment, the expression control element is selected from the group consisting of a promoter, a terminator or an enhancer. In one embodiment, the purification tag is selected from the group consisting of a His tag, a GST tag, an MBP tag, a SUM O tag or a NusA tag. In one embodiment, the vector is an expression vector or a cloning vector, preferably pET-28a (+). pET-28a (+) may comprise a HIS, Thrombin, or T7 protein tag at N-terminus, and an His tag at C-terminus. In the present application, the recombinant collagen may comprise an enzyme cleavage site at the N-terminus to facilitate purification.

In another aspect, provided is a host cell comprising a nucleic acid or a vector described herein. In one embodiment, the host cell is a eukaryotic cell or a prokaryotic cell. In one embodiment, the eukaryotic cell is a yeast cell, an animal cell, and/or an insect cell, and in one embodiment, the prokaryotic cell is an Escherichia coli cell, such as E. coli BL21.

In another aspect, provided is a composition comprising one or more of the recombinant collagens, nucleic acid, vector, and host cell described herein. In one embodiment, the composition is a kit. In one embodiment, the composition is one or more of a biological dressing, a human bionic material, a plastic surgery material or beauty material, an organoid culture material, a cardiovascular stent material, a coating material, a tissue injection filling material, an ophthalmic material, an obstetrics and gynecology biomaterial, a nerve repair and regeneration material, a liver tissue material and blood vessel repair and regeneration material, a 3D printed artificial organ biomaterial, a cosmetic raw material, a pharmaceutical excipient, and a food additive. In one embodiment, the composition is a composition for topical use, injection use, or oral use. In one embodiment, the composition is a composition in the form of a solution, a lyophilized powder, a gel, a sponge, or a fiber.

In another aspect, provided is use of the recombinant collagen, the nucleic acid, the vector, the host cell, and/or the composition of the present application in one or more of a biological dressing, a human bionic material, a plastic surgery or beauty material, an organoid culture material, a cardiovascular stent material, a coating material, a tissue injection filling material, an ophthalmic material, an obstetrics and gynecology biomaterial, a nerve repair and regeneration material, a liver tissue material and blood vessel repair and regeneration material, a 3D printed artificial organ biomaterial, a cosmetic raw material, a pharmaceutical excipient, and a food additive.

In another aspect, provided is a method of promoting cell adhesion, comprising a step of contacting the recombinant collagen, the nucleic acid, the vector, the host cell, and/or the composition of the present application with a cell. In one embodiment, the cell is an animal cell. The animal cell may be a mammalian cell or a human cell. Provided is use of the recombinant collagen, the nucleic acid, the vector, the host cell and/or the composition of the present application in the manufacture of a kit for facilitating cell adhesion.

In another aspect, provided is a beauty method comprising administering the recombinant collagen described herein to a subject, wherein preferably, the administration is a topical administration, oral administration or injection administration, preferably, the subject is a human.

In another aspect, provided is a method of producing the recombinant collagen described herein, comprising:

(1) incubating the host cell described herein under a suitable culture condition;

(2) harvesting the host cell and/or culture medium comprising the recombinant collagen; and

(3) purifying the recombinant collagen.

In one embodiment, the host cell is an E. coli cell, preferably an E. coli BL21 (DE3) cell.

In one embodiment, step (1) comprises culturing the E. coli cell in LB medium and inducing expression with IPTG.

In one embodiment, step (2) comprises harvesting E. coli cells, resuspending them in an equilibrium working solution, homogenizing (preferably high-pressure homogenizing) the E. coli cells, and isolating the supernatant. In one embodiment, the equilibrium working solution comprises 100-500 mM sodium chloride, 10-50 mM Tris, 10-50 mM imidazole, at pH 7-9.

In one embodiment, step (3) comprises crude purification, enzymatic digestion, fine purification, and/or reverse nickel column purification.

In one embodiment, the crude purification comprises Ni-agarose resin column purification of the supernatant to obtain an eluate containing the target protein, wherein the eluate contains 100-500 mM sodium chloride, 10-50 mM Tris, and 100-500 mM imidazole, preferably at pH 7-9.

In one embodiment, fine purification comprises gradient elution of the eluate containing the target protein with a strong anion exchange chromatography column; wherein preferably, the gradient elution comprises 0-15% solution B for 1-5 minutes followed by holding for 3 column volumes, 15-30% solution B for 1-5 minutes followed by holding for 3 column volumes, 30-50% solution B for 1-5 minutes followed by holding for 3 column volumes, 50-100% solution B for 1-5 minutes followed by holding for 3 column volumes; wherein the solution B contains 10-50 mM Tris, 0.5-5 M sodium chloride, at pH 7-9. In one embodiment, step (3) comprises purifying the recombinant collagen with a purification column, such as a nickel column, and/or cleaving the recombinant collagen with a collagen-processing enzyme.

The advantages of the present disclosure include:

1. The recombinant collagen of the present disclosure is derived from human type IV collagen and is recombinant type IV humanized collagen;

2. The recombinant collagen of the present disclosure is suitable for production by E. coli and can be isolated and purified;

3. The recombinant collagen of the present disclosure is produced at high yields and is suitable for subsequent purification (Ni column or strong anion column purification); and

4. The recombinant collagen of the present disclosure has cell adhesion activity. The recombinant collagen of the present disclosure (e.g., C4P7Ch) has higher cell adhesion activity compared to the positive control.

### BRIEF DESCRIPTION OF THE DRAWINGS

FIG. 1 shows the results of electrophoresis detection of C4P7Ca.

FIG. 2 shows the results of electrophoresis detection of C4P7Cb.

FIG. 3 shows the results of electrophoresis detection of C4P7Cc.

FIG. 4 shows the results of electrophoresis detection of C4P7Cd.

FIG. 5 shows the results of electrophoresis detection of C4P7Ce.

FIG. 6 shows the results of electrophoresis detection of C4P7Cf.

FIG. 7 shows the results of electrophoresis detection of C4P7Cg.

FIG. 8 shows the results of electrophoresis detection of C4P7Ch.

FIG. 9 shows the results of electrophoresis detection of C4P7Ea.

FIG. 10 shows the results of electrophoresis detection of C4P7Eb.

FIG. 11 shows the results of electrophoresis detection of C4P7Ec.

FIG. 12 shows the effect of collagen C4P7Ch on cell adhesion.

FIG. 13 shows the effect of collagen C4P7Cf on cell adhesion.

### DETAILED DESCRIPTION OF THE DISCLOSURE

As used herein, a “recombinant collagen” refers to an amino acid sequence encoded by a designed or modified specific gene, or fragment thereof, or a combination of fragments of such functional amino acid sequence, which is prepared by recombinant DNA technology. The gene encoding sequence or amino acid sequence of a recombinant collagen may have a low homology with the gene encoding sequence or amino acid sequence of human collagen. A “recombinant humanized collagen” refers to the full-length or partial amino acid sequence fragment encoded by a specific type gene of human collagen prepared by recombinant DNA technology, or a combination containing functional fragments of human collagen. Herein, the recombinant collagen comprises one or more repeating units. The repeating unit may be derived from human type IV collagen. Thus, the recombinant collagen may be recombinant type IV humanized collagen. The plurality of repeating units may be linked via a linker, which may be a natural amino acid residue(s), for example 1-50 amino acid residues, of the repeating units on human type IV collagen. A repeating unit may be SEQ ID NO: 1, 4, 7, 10, 13, 16, 19, 22, 25, 28, 31, or 33. A recombinant collagen may be SEQ ID NO: 2, 5, 8, 11, 14, 17, 20, 23, 26, 29, or 32.

As used herein, the term “variant” means a recombinant collagen having cell adhesion activity that includes alterations/mutations (i.e., substitutions, additions, insertions, and/or deletions) at one or more positions. Substitution means the replacement of an amino acid occupying a certain position with a different amino acid; deletion means the removal of an amino acid occupying a certain position; and insertion means the addition of an amino acid adjacent to and immediately after an amino acid occupying a certain position. Addition refers to the addition of one or more amino acid residues to the C-terminus and/or N-terminus of an amino acid sequence. The substitution may be a conservative substitution. A variant of a repeating unit may be a sequence in which one or more amino acid residues in SEQ ID NO: 1, 4, 7, 10, 13, 16, 19, 22, 25, 28, 31 or 33 are changed or mutated (i.e., substituted, added, inserted and/or deleted). A variant of a recombinant collagen may be a sequence in which one or more amino acid residues in SEQ ID NO: 2, 5, 8, 11, 14, 17, 20, 23, 26, 29 or 32 are changed or mutated (i.e., substituted, added, inserted and/or deleted).

For example, a variant of a repeating unit of SEQ ID NO: 1 may be a variant comprising the following mutations: the addition of Gfpgfp (SEQ ID NO: 34) or the N-terminal truncated fragment of SEQ ID NO: 34 (for example 1-5 amino acid residues, such as 1, 2, 3, 4 or 5 amino acid residues are truncated from the N-terminus of SEQ ID NO: 34, corresponding to fpgfp (SEQ ID NO: 58), pgfp (SEQ ID NO: 59), gfp, fp and p residues, respectively) to the N-terminus of the amino acid sequence of SEQ ID NO: 1, and/or the addition of GFMGPPGPQGQPGLP (SEQ ID NO: 35) or a C-terminal truncated fragment of SEQ ID NO: 35 (1-14 amino acid residues, e.g., 1, 2, 3, 4, 5, 6, 7, 8, 9, 10, 11, 12, 13 or 14 amino acid residues, are truncated from the C-terminus of SEQ ID NO: 35) to the C-terminus of the amino acid sequence of SEQ ID NO: 1. A variant of a repeating unit of SEQ ID NO: 1 may be a variant comprising the following mutations: the addition of Gfpgfp (SEQ ID NO: 34) or the N-terminal truncated fragment thereof (for example 1, 2, 3, 4 or 5 amino acid residues are truncated from the N-terminus of SEQ ID NO: 34) to the N-terminus of the amino acid sequence of SEQ ID NO: 1, and/or truncation of 1-5 amino acid residues, such as 1, 2 or 3 amino acid residues, at the C-terminus of the amino acid sequence of SEQ ID NO: 1.

A variant of a repeating unit of SEQ ID NO: 28 may be a variant comprising the following mutations: the addition of Glpgtp (SEQ ID NO: 36) or the N-terminal truncated fragment thereof (for example 1-5 amino acid residues, e.g., 1, 2, 3, 4 or 5 amino acid residues are truncated from the N-terminus of SEQ ID NO: 36) to the N-terminus of the amino acid sequence of SEQ ID NO: 28.

In the context of the present disclosure, a conservative substitution may be defined by substitution within one or more of the amino acid classes reflected in one or more of the following tables:

Conservative classes of amino acid residues:

| | |
|---|---|
| Acidic residues | D and E |
| Basic residues | K, R and H |
| Hydrophilic uncharged residues | S, T, N, and Q |
| Aliphatic uncharged residues | G, A, V, L and I |
| Non-polar uncharged residues | C, M and P |
| Aromatic residues | F, Y , and W. |

Alternative physical and functional classification of amino acid residues:

| | |
|---|---|
| Alcohol group-containing residues | S and T |
| Aliphatic Residues | I, L, V and M |
| Cycloalkenyl-related residues | F, H, W and Y |
| Hydrophobic residues | A, C, F, G, H, I, L, M , |
| | R, T, V, W and Y |
| Negatively charged residues | D and E |
| Polar residues | C, D, E, H, K, N, Q, R, S and T |
| Positively charged residues | H, K and R |
| Small residues | A, C, D, G, N, P, S, T and V |
| Minimal residues | A, G and S |
| Residues involved in | A, C, D, E, G, H, K, N, |
| turn formation | Q, R, S, P and T |
| Flexible residues | Q, T, K, S, G, P, D, E, and R. |

As used herein, “cell adhesion” refers to the adhesion between cells and collagen. Collagen, such as the recombinant collagen described herein, can facilitate adhesion between cells and the container in which the cells are cultured.

As used herein, the term “expression” includes any step involved in the production of recombinant collagen, including, but not limited to, transcription, post-transcriptional modification, translation, post-translational modification, and secretion.

As used herein, the term “expression vector” means a linear or circular DNA molecule comprising a polynucleotide which encodes a recombinant collagen protein and is operably linked to a control sequence provided for its expression.

As used herein, the term “host cell” means any cell type amenable to transformation, transfection, transduction, etc. with a nucleic acid construct or expression vector comprising a polynucleotide of the present disclosure. The term “host cell” encompasses any progeny of a parent cell that is not identical to the parent cell due to a mutation occurring during replication.

As used herein, the term “nucleic acid” means a single-stranded or double-stranded nucleic acid molecule that is isolated from a naturally occurring gene, or is modified in a manner which is otherwise not present in nature to contain a segment of a nucleic acid, or is synthetic, and the nucleic acid molecule may comprise one or more control sequences. The nucleic acid may be SEQ ID NO: 3, 6, 9, 12, 15, 18, 21, 24, 27, 30 or 33. The nucleic acid may be a codon-optimized nucleic acid, for example, a nucleic acid that is codon-optimized for expression in E. coli cells.

The term “operably linked” means a configuration in which a control sequence is placed at an appropriate position relative to an encoding sequence of a polynucleotide such that the control sequence directs the expression of the encoding sequence.

The degree of association between two amino acid sequences or between two nucleotide sequences is described by the parameter “sequence identity”. For the purposes of this disclosure, the sequence identity between two amino acids is determined by Needleman-Wunsch Algorithm implemented by the Needle program (Needleman and Wunsch, 1970, J. Mol. Biol. 48:443-453) of the EMBOSS software package (EM BOSS: The European Molecular Biology Open Software Suite, Rice et al, 2000, Trends Genet. 16:276-277) (Version 5.0.0 or later is preferred). The parameters used are the gap opening penalty of 10, the gap extension penalty of 0.5, and the EBLOSUM 62 (EM BOSS version of BLOSUM 62) substitution matrix. Needle's output labeled “Longest Identity” (obtained using the non-simplified option) is used as the percent identity and calculated as follows: (identical residues×100)/(alignment length−total number of gaps in the alignment)

For the purposes of this disclosure, the sequence identity between two deoxynucleotide sequences is determined by the Needleman-Wunsch Algorithm implemented by the Needle program (Needleman and Wunsch, 1970) of the EM BOSS software package (EM BOSS: The European Molecular Biology Open Software Suite, Rice et al, 2000) (Version 5.0.0 or later is preferred). The parameters used are the gap opening penalty of 10, the gap extension penalty of 0.5, and the EDNAFULL (EMBOSS version of NCBI NUC4.4) substitution matrix. Needle's output labeled “Longest Identity” (obtained using the non-simplified option) is used as the percent identity and calculated as follows: (identical deoxyribonucleotides×100)/(alignment length−total number of gaps in the alignment)

### Recombinant Collagen

The present disclosure provides a recombinant collagen comprising one or more repeating units linked directly or via a linker, wherein the repeating units comprise an amino acid sequence selected from SEQ ID NO: 1, 4, 7, 10, 13, 16, 19, 22, 25, 28, 31 or 33, or a variant thereof. The variant may be (1) an amino acid sequence in which one or more amino acid residues are mutated in the amino acid sequence of SEQ ID NO: 1, 4, 7, 10, 13, 16, 19, 22, 25, 28, 31 or 33, or (2) an amino acid sequence having at least 80%, 85%, 86%, 87%, 88%, 89%, 90%, 91%, 92%, 93%, 94%, 95%, 96%, 97%, 98%, 99%, or 100% identity with the amino acid sequence of SEQ ID NO: 1, 4, 7, 10, 13, 16, 19, 22, 25, 28, 31, or 33. With respect to the recombinant collagen described herein, the mutation may be selected from the group consisting of a substitution, addition, insertion, or deletion. Preferably, the substitution is a conservative amino acid substitution.

The recombinant collagen described herein may comprise a plurality of repeating units, for example, 2-50 repeating units, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 repeating units.

The linker in the recombinant collagen described herein may comprise one or more amino acid residues, for example, 1-10, 1-9, 1-8, 1-7, 1-6, 1-5, 1-4, 1-3, or 1-2 amino acid residues.

The recombinant collagen is recombinant human type IV collagen or recombinant type IV humanized collagen, preferably having cell adhesion activity. The recombinant collagen described herein may be human-derived, and thus is a recombinant type IV humanized collagen.

The recombinant collagen described herein may also comprise an amino acid sequence selected from the group consisting of SEQ ID NO: 2, 5, 8, 11, 14, 17, 20, 23, 26, 29 or 32, or a variant thereof, wherein the variant is (1) an amino acid sequence having one or more amino acid residue mutations in said amino acid sequence or (2) an amino acid sequence having at least 80%, 85%, 86%, 87%, 88%, 89%, 90%, 91%, 92%, 93%, 94%, 95%, 96%, 97%, 98%, 99% or 100% identity to said amino acid sequence.

### Nucleic Acid Constructs

The present disclosure also relates to a nucleic acid construct comprising a nucleic acid of the present disclosure operably linked to one or more control sequences that direct the expression of an encoding sequence in a suitable host cell under a condition compatible with the control sequences. The vector may comprise a nucleic acid construct.

Nucleic acids can be modified in a variety of ways to enable the expression of recombinant collagen. Depending on the expression vector, it may be desirable or necessary to modify the nucleic acid prior to its insertion into the vector. Techniques for modifying nucleic acids using recombinant DNA methods are well known in the art.

The control sequence may be a promoter, i.e., it is recognized by a host cell for the expression of a polynucleotide encoding a recombinant collagen of the present disclosure. The promoter comprises transcriptional control sequences that mediate the expression of recombinant collagen. The promoter can be any nucleic acid that exhibits transcriptional activity in a host cell, including variants, truncated or hybrid promoters, and can be obtained from a gene encoding an extracellular or intracellular recombinant collagen homologous or heterologous to the host cell.

Examples of suitable promoters for directing transcription of the vectors or nucleic acid constructs of the disclosure in bacterial host cells are promoters obtained from: Bacillus amyloliquefaciens alpha-amylase gene (amyQ), Bacillus licheniformis alpha-amylase gene (amyL), Bacillus licheniformis penicillinase gene (penP), Bacillus stearothermophilus maltoamylase gene (amyM), Bacillus subtilis fructan sucrase gene (sacB), Bacillus subtilis xylA and xyIB genes, Bacillus thuringiensis cryIIIA gene, E. coli lac operon, and E. coli trc promoter.

In yeast hosts, useful promoters are obtained from the following genes: Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triosephosphate isomerase (TPI), Saccharomyces cerevisiae metallothionein (CUP1), and Saccharomyces cerevisiae 3-phosphoglycerate kinase.

The control sequence may also be a transcription terminator recognized by the host cell to terminate transcription. The terminator may be operably linked to the 3′ end of the polynucleotide encoding the recombinant collagen. A ny terminator that is functional in the host cell may be used in the present disclosure.

Preferred terminators for bacterial host cells are obtained from the genes of Bacillus clausii alkaline protease (aprH), Bacillus licheniformis alpha-amylase (amyL), and E. coli ribosomal RNA (rrnB).

Preferred terminators for yeast host cells are obtained from the following genes: Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al. (1992).

The control sequence may also be an mRNA stabilizer region downstream of the promoter and upstream of the encoding sequence of the gene, which increases the expression of the gene.

Examples of suitable mRNA stabilizer regions are obtained from the following genes: Bacillus thuringiensis cryllIA gene (WO 94/25612) and Bacillus subtilis SP82 gene (Hue et al., 1995, Journal of Bacteriology 177:3465-3471).

The control sequence may also be a leader sequence, i.e., a non-translated region of an mRNA that is important for translation in the host cell. The leader sequence is operably linked to the 5′ end of the polynucleotide encoding the recombinant collagen. Any leader sequence that is functional in the host cell may be used.

Suitable leader sequences for yeast host cells are obtained from the following genes: Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, which is operably linked to the 3′ end of the polynucleotide and when transcribed, is recognized by the host cell as a signal to add polyadenylate residues to the transcribed mRNA. Any polyadenylation sequence that is functional in the host cell may be used.

Useful polyadenylation sequences for yeast host cells are described by Guo and Sherman, 1995, Mol. Cellular Biol. 15:5983-5990.

The control sequence may also be a signal peptide encoding region that encodes a signal peptide linked to the N-terminus of the recombinant collagen and directs the recombinant collagen into the secretory pathway of the cell. The 5′-end of the encoding sequence of the polynucleotide may itself contain a signal peptide encoding sequence that is naturally linked in open reading frame to the segment of the encoding sequence encoding the recombinant collagen. Alternatively, the 5′-end of the encoding sequence may contain a signal peptide encoding sequence that is foreign to the encoding sequence. In cases where the encoding sequence does not naturally contain a signal peptide encoding sequence, a foreign signal peptide encoding sequence may be required. A Iternatively, the foreign signal peptide encoding sequence may simply replace the natural signal peptide encoding sequence in order to enhance the secretion of the recombinant collagen. However, any signal peptide encoding sequence that directs the expressed recombinant collagen into the secretory pathway of the host cell may be used.

An effective signal peptide encoding sequence for a bacterial host cell is a signal peptide encoding sequence obtained from the following genes: Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral protease (nprT, nprS, nprM) and Bacillus subtilis prsA. Additional signal peptides are described by Simonen and Palva, 1993, Microbiological Reviews 57:109-137.

Useful signal peptides for yeast host cells are obtained from the following genes: Saccharomyces cerevisiae alpha-factor and Saccharomyces cerevisiae invertase. Other useful signal peptide encoding sequences are described by Romanos et al. (Yeast 8:423-488).

### Expression Vector

The disclosure also relates to recombinant expression vectors comprising a nucleic acid, a promoter, and transcription and translation termination signals of the present disclosure. Nucleic acids and control sequences may be ligated together to produce recombinant expression vectors that may include one or more convenient restriction sites for insertion or substitution of a polynucleotide encoding the recombinant collagen at such sites. Alternatively, the polynucleotide may be expressed by inserting a nucleic acid, or a nucleic acid construct comprising the nucleic acid into an appropriate vector for expression. When an expression vector is produced, the encoding sequence is located in the vector such that the encoding sequence is operably linked to an appropriate control sequence for expression.

A recombinant expression vector may be any vector (e.g., a plasmid or virus) that can be conveniently subjected to a recombinant DNA procedure and may enable the expression of a polynucleotide. The choice of the vector will typically depend on the compatibility of the vector with the host cell into which the vector is to be introduced. The vector may be a linear chain or a closed circular plasmid.

The vector may be an autonomously replicating vector, i.e. a vector that exists as an extrachromosomal entity, whose replication is independent of chromosomal replication, such as a plasmid, an extrachromosomal element, a minichromosome or an artificial chromosome. The vector may contain any means for ensuring self-replication. Alternatively, the vector may be one that, when introduced into a host cell, integrates into the genome and replicates with one or more chromosomes into which it has been integrated. Furthermore, separate vectors or plasmids or two or more vectors or plasmids, which collectively contain the total DNA to be introduced into the genome of the host cell may be used, or transposons may be used.

The vector preferably contains one or more selectable markers that allow for convenient selection of transformed cells, transfected cells, transduced cells and the like. A selectable marker is a gene whose product provides a biocide resistance or a virus resistance, a resistance to heavy metals, a prototrophy to auxotroph, etc.

Examples of bacterial selectable markers are the dal gene from Bacillus licheniformis or Bacillus subtilis, or markers conferring antibiotic resistance, such as ampicillin, chloramphenicol, kanamycin, neomycin, spectinomycin, or tetracycline resistance. Suitable markers for yeast host cells include, but are not limited to: ADE2, HIS3, LEU2, LYS2, MET3, TRP1, and URA 3.

The selectable marker may be a dual selectable marker system as described in WO 2010/039889. In one aspect, the dual selectable marker is an hph-tk dual selectable marker system.

The vector may contain an element that allows integration of the vector into the genome of the host cell or autonomous replication of the vector in the cell independent of the genome.

For integration into the host cell genome, the vector may rely on the polynucleotide sequence encoding the recombinant collagen or any other element of the vector for integration into the genome by homologous or non-homologous recombination. Alternatively, the vector may contain an additional polynucleotide for directing integration by homologous recombination into the genome of the host cell at a precise location(s) in the chromosome(s). To increase the likelihood of integration at a precise location, the integrational element should contain a sufficient number of nucleic acids, such as 100 to 10,000 base pairs, 400 to 10,000 base pairs, and 800 to 10,000 base pairs, which have a high degree of sequence identity to the corresponding target sequences to enhance the probability of homologous recombination. The integrational element may be any sequence homologous to a target sequence within the genome of the host cell. Furthermore, the integrational element may be a non-encoding or encoding polynucleotide. In another aspect, the vector can be integrated into the genome of the host cell by non-homologous recombination.

In order to replicate autonomously, the vector may further comprise an origin of replication enabling the vector to replicate autonomously in the host cell in question. The origin of replication may be any plasmid replicator that functions in cells to mediate autonomous replication. The term “origin of replication” or “plasmid replicator” means a polynucleotide that enables a plasmid or vector to replicate in vivo.

Examples of bacterial origins of replication are the origins of replication of plasmids pBR 322, pUC19, pA CY C177, and pA CY C184 that allow replication in E. coli, as well as the origins of replication of plasmids pU B 110, pE 194, pTA 1060, and pA M B1 that allow replication in Bacillus.

Examples of origins of replication for use in yeast host cells are 2-micron origin of replication, ARS1, ARS4, the combination of ARS1 and CEN 3, as well as the combination of ARS4 and CEN6.

More than one copy of the polynucleotide of the present disclosure can be inserted into a host cell to increase the production of recombinant collagen. An increased number of copies of a polynucleotide can be obtained by integrating at least one additional copy of the sequence into the host cell genome or by including an amplifiable selectable marker gene together with the polynucleotide where cells comprising amplified copies of the selectable marker gene and thus additional copies of the polynucleotide can be selected by culturing the cells in the presence of an appropriate selectable agent.

Procedures for ligating the elements described above to construct the recombinant expression vectors of the present disclosure are well known to those of ordinary skill in the art (see, for example, Sambrook et al., 1989, Molecular Cloning: A Laboratory Manual (2nd ed., Cold Spring Harbor Laboratory Press, Cold Spring Harbor, NY)).

### Host Cell

The present disclosure also relates to recombinant host cells comprising a polynucleotide of the present disclosure operably linked to one or more control sequences that direct the production of the recombinant collagen of the present disclosure. The construct or vector comprising the polynucleotide is introduced into the host cell such that the construct or vector is maintained as a chromosomal integrant or as an autonomously replicating extrachromosomal vector, as described earlier. The term “host cell” encompasses any progeny of a parent cell that is not identical to the parent cell due to a mutation occurring during replication. The choice of host cell will depend largely on the gene encoding the recombinant collagen and its source.

The host cell may be any cell useful in the recombinant production of the recombinant collagen of the present disclosure, for example, a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, Escherichia coli, Flavobacterium, Fusobacterium, Helicobacter, Ilyobacter, Neisseria, Pseudomonas, Salmonella, and Ureaplasma.

The host cell may also be a cell of a eukaryotic organism, such as a mammalian, insect, plant or fungus. Plant cells herein do not include plant cells that can regenerate into a whole plant. Animal cells also do not include cells that can generate an animal body.

The host cells may be fungal cells such as Basidiomycota, Chytridiomycota, Zygomycota, and Oomycota, among others. The host cell may be a yeast cell, including ascosporogenous yeast (Endomycetales), basidiosporogenous yeast, and yeast belonging to Fungi Imperfecti (Blastomycetes). The yeast host cell may be a cell of Candida, Hansenula, Kluyveromyces, Pichia, Saccharomyces, Schizosaccharomyces or Yarrowia, such as a cell of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis or Yarrowia lipolytica.

### Production Method

The present disclosure also relates to a method of producing the recombinant collagen as described herein, comprising:

(1) incubating the host cell described herein under a suitable culture condition;

(2) harvesting the host cell and/or culture medium comprising the recombinant collagen; and

(3) purifying the recombinant collagen.

Using methods known in the art, the host cells are cultured in a suitable nutrient medium for the production of recombinant collagen. For example, cells can be cultured in shake flask, or by small-or large-scale fermentation (including continuous, batch, fed-batch or solid-state fermentation) in a laboratory or industrial fermenter, wherein the cultivation is performed in a suitable medium and under conditions that allow expression and/or isolation of the recombinant collagen. The cultivation is performed in a suitable nutrient medium comprising carbon and nitrogen sources and inorganic salts using procedures known in the art. Suitable media may be obtained from commercial suppliers or may be prepared according to disclosed compositions (e.g., in the catalogs of American Type Culture Collection). If a recombinant collagen is secreted into the nutrient medium, the recombinant collagen can be recovered directly from the medium. If a recombinant collagen is not secreted, it can be recovered from the cell lysate.

Recombinant collagen can be detected using methods known in the art that are specific for recombinant collagen. These detection methods include, but are not limited to, the use of specific antibodies. For example, an adhesion assay can be used to determine the activity of the recombinant collagen.

The recombinant collagen can be recovered by methods known in the art. For example, the recombinant collagen can be recovered from the nutrient medium by conventional procedures including, but not limited to, collection, centrifugation, filtration, extraction, spray drying, evaporation, or precipitation. In one aspect, the recombinant collagen is recovered from the fermentation broth comprising recombinant collagen.

Recombinant collagen can be purified by known procedures in the art, including, but not limited to, chromatography (e.g., ion exchange chromatography, affinity chromatography, hydrophobic chromatography, focusing chromatography, and size exclusion chromatography), electrophoretic procedures (e.g., preparative isoelectric focusing electrophoresis), differential solubility (e.g., ammonium sulfate precipitation), SDS-PAGE, or extraction to obtain substantially pure recombinant collagen.

Step (1) may comprise one or more of the following steps: constructing an expression plasmid, e.g., inserting an encoding nucleotide sequence into a pET-28a-Trx-His expression vector to obtain a recombinant expression plasmid. Successfully constructed expression plasmids can be transformed into E. coli cells (e.g., E. coli competent cells BL21 (DE3)). The specific process can be as follows: (1) taking the plasmid to be transformed and adding it to E. coli competent cells BL21 (DE3); (2) placing the mixture on ice in an ice bath (e.g., for 10-60 min, e.g., 30 min), followed by a heat shock in a water bath (e.g., at 40-50° C., e.g., 42° C., for 45-90 s), taking out the mixture and placing it on ice in an ice bath (e.g., for 1-5 min, e.g., 2 min); (3) adding liquid LB medium followed by an incubation (e.g., at 35-40° C., e.g., 37° C., at 150-300 rpm, e.g., 220 rpm for 40-80 min, e.g., 60 min); (4) spreading the bacterial solution and picking single colonies. For example, the bacterial solution is taken and evenly spread on LB plates containing ampicillin sodium, and the plates are cultured in an incubator at 37°° C. for 15-17 hours until uniform-sized colonies grow in the plates.

Step (2) may comprise culturing the single colonies in LB medium containing an antibiotic stock (e.g., in a shaker at 150-300 rpm, e.g., 220 rpm, at a constant temperature of 35-40° C., e.g., 37° C. for 5-10 hours, e.g., 7 hours). Then, the cultured shake flask is cooled to 10-20°° C., for example 16° C., and IPTG is added to induce expression for a period of time, and then the cells are collected (for example, by centrifugation).

Step (3) may include resuspending the bacterial cells with an equilibrium working solution, cooling the bacterial liquid to ≤15° C., performing homogenization (e.g., high-pressure homogenization, e.g., 1-5 times, e.g., 2 times), and separating the homogenized bacterial liquid to obtain a supernatant. The equilibrium working solution may comprise 100-500 mM sodium chloride, 10-50 mM Tris, and 10-50 mM imidazole, at pH 7-9. For example, the concentration of sodium chloride may be 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, or 490 nM. The concentration of Tris may be 10, 15, 20, 25, 30, 35, 40, 45, or 50 nM. The concentration of imidazole may be 10, 15, 20, 25, 30, 35, 40, 45, or 50 nM. The pH may be 7, 7.5, 8, 8.5, or 9.

Step (3) may include purifying and enzymatically digesting the recombinant collagen. The purification may be a crude purification comprising a Ni-agarose column purification of the supernatant to obtain an eluate containing the target protein. Crude purification may comprise washing the column material with water, for example for 2-10 column volumes (CVs), for example 5 CVs. The column material can be equilibrated with an equilibration solution (200 mM sodium chloride, 25 mM Tris, 20 mM imidazole at pH 8.0), for example for 2-10 CVs, for example 5 CVs. The equilibrium solution may comprise 100-500 mM sodium chloride, 10-50 mM Tris, and 10-50 mM imidazole at pH 7-9. For example, the concentration of sodium chloride may be 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, or 490 nM. The concentration of Tris may be 10, 15, 20, 25, 30, 35, 40, 45, or 50 nM. The concentration of imidazole may be 10, 15, 20, 25, 30, 35, 40, 45, or 50 nM. The pH may be 7, 7.5, 8, 8.5, or 9.

Step (3) may comprise loading the supernatant onto the column material and washing the impurity proteins with a washing solution. The washing solution may comprise 100-500 mM sodium chloride, 10-50 mM Tris, and 10-50 mM imidazole at pH 7-9. For example, the concentration of sodium chloride may be 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, or 490 nM. The concentration of Tris may be 10, 15, 20, 25, 30, 35, 40, 45, or 50 nM. The concentration of imidazole may be 10, 15, 20, 25, 30, 35, 40, 45, or 50 nM. The pH may be 7, 7.5, 8, 8.5, or 9. Then, the eluent can be added and the flow-through liquid is collected. The eluent may comprise 100-500 mM sodium chloride, 10-50 mM Tris, 100-500 mM imidazole, at pH 8.0. For example, the concentration of sodium chloride may be 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, or 490 nM. The concentration of Tris may be 10, 15, 20, 25, 30, 35, 40, 45, or 50 nM. The concentration of imidazole may be 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, or 490 nM. The pH may be 7, 7.5, 8, 8.5, or 9.

The enzymatic digestion may comprise adding a collagen-processing enzyme for enzymatic digestion (at a ratio of the total amount of protein to the total amount of collagen-processing enzyme of 10-100:1, e.g., 50:1, at 10-20° C., e.g., 16°° C. for 2-8 h, e.g., 4 h). The enzymatically digested protein solution is dialyzed, for example, in a dialysis bag at 1-6° C., for example 4° C. for 1-8 hours, for example 2 hours, and then transferred to a new dialysate for dialysis at 1-6° C., for example 4°° C. overnight.

Purification may include fine purification (e.g., for proteins with an isoelectric point >8.0). Preferably, the fine purification comprises using a strong anion exchange chromatography column (e.g., the pH may be 7, 7.5, 8, 8.5 or 9.) The eluate containing the target protein or the product after enzymatic digestion (e.g., enzymatically digested and dialyzed product) is subjected to gradient elution. The gradient elution comprises 0-15% solution B for 1-5 minutes followed by holding for 1-5 column volumes, e.g., 3 column volumes, 15-30% solution B for 1-5 minutes followed by holding for 1-5 column volumes, e.g., 3 column volumes, 30-50% solution B for 1-5 minutes followed by holding for 1-5 column volumes, e.g., 3 column volumes, 50-100% solution B for 1-5 minutes followed by holding for 1-5 column volumes, e.g., 3 column volumes. Solution B may comprise 10-50 mM Tris, 0.5-5 M sodium chloride, at pH 7-9. For example, the concentration of Tris is 15, 20, 25, 30, 35, 40, or 45 mM. The concentration of sodium chloride is 1, 2, 3, or 4 M. The pH may be 7, 7.5, 8, 8.5, or 9. Fine purification may include equilibrating the column material with solution A and loading, followed by gradient elution. Solution A may contain 10-50 mM Tris, 10-50 mM sodium chloride, at pH 7-9. For example, the concentration of Tris is 15, 20, 25, 30, 35, 40, or 45 mM. The concentration of sodium chloride is 15, 20, 25, 30, 35, 40 or 45 mM. The pH may be 7, 7.5, 8, 8.5, or 9.

To clarify the purpose, technical solutions, and advantages of the present disclosure, the technical solutions in the examples will be clearly and completely described below in conjunction with the following examples. Obviously, the described examples are partial embodiments of the present disclosure, rather than all of the embodiments. Based on the examples of the present disclosure, all other examples obtained by ordinary technicians in the art without making any creative work fall within the scope of protection of the present disclosure.

The following examples are further provided to illustrate the present disclosure.

### EXAMPLES

The present disclosure is further illustrated by the following examples, but any example or combination thereof should not be construed as limiting the scope of the present disclosure. The scope of the present disclosure is defined by the following claims. As a person skilled in the art combines this specification with common knowledge in the art, they can clearly discern the scope defined by the claims. Without departing from the spirit and scope of the present disclosure, those skilled in the art can make modifications to the technical solution of the present disclosure. Such modifications also fall within the scope of the present disclosure.

### Example 1: Construction, Expression and Screening of Recombinant Type IV

### Humanized Collagen Fragments

1. Large-scale screening for functional regions was carried out to obtain the following different target gene functional regions of recombinant type IV humanized collagen.

1) The amino acid sequence of C4P7Ch (the amino acid sequence of repeating unit is Gakgdkgskgevgfpglagspgipgskgeq, SEQ ID NO: 1, and the number of repeating units is 10; the amino acid sequence of C4P7Ch is SEQ ID NO: 2):

(SEQ ID NO: 2)
Gakgdkgskgevgfpglagspgipgskgeq
Gakgdkgskgevgfpglagspgipgskgeq
Gakgdkgskgevgfpglagspgipgskgeq
Gakgdkgskgevgfpglagspgipgskgeq
Gakgdkgskgevgfpglagspgipgskgeq
Gakgdkgskgevgfpglagspgipgskgeq
Gakgdkgskgevgfpglagspgipgskgeq
Gakgdkgskgevgfpglagspgipgskgeq
Gakgdkgskgevgfpglagspgipgskgeq
Gakgdkgskgevgfpglagspgipgskgeq
C4P7Ch base sequence (SEQ ID NO: 3):
(SEQ ID NO: 3)
GGGGCTAAAGGAGACAAGGGCAGCAAGGGCGAAGTCGGTTTCCCA
GGTCTGGCTGGTAGCCCGGGCATCCCGGGTTCAAAGGGTGAACAA
GGTGCTAAAGGCGACAAAGGCAGCAAGGGTGAGGTTGGTTTCCCG
GGTCTGGCGGGTTCTCCAGGCATCCCGGGTAGCAAAGGAGAACAA
GGTGCGAAAGGCGATAAAGGCTCCAAGGGTGAAGTGGGCTTCCCG
GGTTTAGCCGGTAGCCCAGGTATTCCGGGTAGCAAAGGCGAACAG
GGTGCGAAAGGCGACAAAGGGAGTAAGGGCGAGGTGGGTTTTCCG
GGTTTGGCTGGCTCGCCGGGTATTCCGGGTTCAAAGGGCGAACAG
GGCGCGAAAGGTGATAAAGGCAGCAAAGGCGAGGTTGGCTTCCCG
GGTCTGGCAGGTAGCCCGGGTATCCCGGGTAGCAAGGGTGAGCAG
GGTGCCAAAGGCGACAAAGGTAGCAAGGGGGAAGTGGGTTTTCCG
GGACTGGCAGGTAGCCCGGGTATCCCGGGTTCTAAGGGCGAGCAG
GGTGCGAAAGGTGACAAAGGTAGCAAGGGCGAGGTTGGCTTTCCG
GGCTTGGCGGGTAGCCCGGGCATTCCGGGCTCCAAGGGTGAACAA
GGTGCGAAAGGTGATAAAGGCTCTAAGGGTGAGGTTGGTTTTCCG
GGTCTGGCGGGTTCCCCGGGCATTCCGGGCTCGAAGGGCGAGCAA
GGTGCTAAAGGTGATAAGGGCTCCAAGGGCGAGGTGGGTTTCCCG
GGCCTGGCAGGCTCTCCGGGCATCCCGGGTTCGAAGGGCGAACAG
GGTGCGAAAGGCGATAAAGGTTCCAAGGGCGAAGTCGGATTCCCT
GGCCTCGCCGGTAGCCCGGGCATCCCTGGCTCCAAGGGCGAGCAG

2) C4P7Cf (the amino acid sequence of repeating unit is Gakgdkgskgevgfpglagspgipgskgeqgfmgppgpq, SEQ ID NO: 4, and the number of repeating units is 8; the amino acid sequence of C4P7Cf is SEQ ID NO: 5):

(SEQ ID NO: 5)
Gakgdkgskgevgfpglagspgipgskgeqgfmgppgpq
Gakgdkgskgevgfpglagspgipgskgeqgfmgppgpq
Gakgdkgskgevgfpglagspgipgskgeqgfmgppgpq
Gakgdkgskgevgfpglagspgipgskgeqgfmgppgpq
Gakgdkgskgevgfpglagspgipgskgeqgfmgppgpq
Gakgdkgskgevgfpglagspgipgskgeqgfmgppgpq
Gakgdkgskgevgfpglagspgipgskgeqgfmgppgpq
Gakgdkgskgevgfpglagspgipgskgeqgfmgppgpq
C4P7Cf base sequence (SEQ ID NO: 6):
(SEQ ID NO: 6)
GGAGCTAAAGGGGACAAAGGCTCCAAAGGCGAAGTCGGTTTCCCGG
GCCTGGCGGGTAGCCCGGGTATCCCGGGTAGCAAGGGTGAGCAGG
GGTTCATGGGTCCACCGGGCCCACAGGGTGCCAAAGGTGATAAAG
GTTCTAAGGGCGAGGTGGGTTTCCCGGGGCTGGCGGGTTCTCCGG
GCATTCCGGGAAGCAAGGGTGAACAGGGCTTTATGGGTCCGCCAG
GTCCGCAGGGTGCGAAAGGTGATAAAGGCAGCAAGGGAGAAGTTG
GCTTCCCGGGCCTGGCAGGCAGCCCGGGCATTCCGGGGTCGAAGG
GCGAACAAGGTTTCATGGGTCCGCCTGGTCCGCAAGGTGCGAAAG
GTGATAAGGGTAGCAAGGGTGAAGTGGGTTTTCCGGGATTAGCGG
GTTCTCCGGGCATTCCGGGTTCAAAAGGTGAACAAGGCTTTATGG
GTCCGCCTGGCCCGCAGGGTGCTAAAGGCGACAAGGGTAGCAAAG
GCGAGGTAGGTTTCCCGGGTTTGGCGGGCAGCCCGGGCATTCCGG
GTTCCAAGGGCGAGCAGGGTTTTATGGGCCCACCGGGCCCGCAAG
GCGCAAAAGGTGATAAGGGCAGCAAAGGCGAGGTGGGCTTCCCGG
GACTGGCAGGTTCTCCGGGTATCCCGGGTTCCAAGGGTGAGCAGG
GTTTCATGGGCCCACCGGGTCCGCAGGGTGCGAAAGGCGACAAAG
GTAGCAAGGGCGAAGTTGGTTTTCCGGGCCTGGCTGGTTCGCCGG
GCATCCCGGGCTCCAAGGGCGAGCAAGGCTTCATGGGTCCACCGG
GTCCGCAAGGTGCCAAAGGCGACAAAGGTAGCAAGGGCGAGGTTG
GTTTTCCGGGCTTGGCTGGTAGCCCTGGCATCCCGGGGTCCAAGG
GTGAACAGGGCTTTATGGGTCCGCCGGGCCCTCAA

3) C4P7Ca amino acid sequence (the amino acid sequence of the repeating unit is GFPGFPGAKGDKGSKGEVGFPGLAGSPGIPGSKGEQGFMGPPGPQGQPGLP, SEQ ID NO: 7, and the number of repeating units is 6; the amino acid sequence of C4P7Ca is SEQ ID NO: 8):

(SEQ ID NO: 8)
GFPGFPGAKGDKGSKGEVGFPGLAGSPGIPGSKGEQGFMGPPGPQGQPGLP
GFPGFPGAKGDKGSKGEVGFPGLAGSPGIPGSKGEQGFMGPPGPQGQPGLP
GFPGFPGAKGDKGSKGEVGFPGLAGSPGIPGSKGEQGFMGPPGPQGQPGLP
GFPGFPGAKGDKGSKGEVGFPGLAGSPGIPGSKGEQGFMGPPGPQGQPGLP
GFPGFPGAKGDKGSKGEVGFPGLAGSPGIPGSKGEQGFMGPPGPQGQPGLP
GFPGFPGAKGDKGSKGEVGFPGLAGSPGIPGSKGEQGFMGPPGPQGQPGLP
C4P7Ca base sequence (SEQ ID NO: 9):
(SEQ ID NO: 9)
GGATTTCCCGGGTTCCCGGGTGCCAAAGGGGATAAAGGTTCAAAG
GGCGAAGTGGGTTTCCCGGGTTTGGCTGGTAGCCCGGGTATCCCG
GGTAGCAAAGGCGAACAGGGCTTTATGGGTCCGCCAGGACCGCAG
GGTCAACCGGGACTGCCGGGTTTTCCGGGCTTCCCGGGTGCGAAA
GGCGATAAAGGTTCCAAGGGTGAAGTTGGTTTTCCGGGTCTTGCA
GGCAGCCCGGGTATTCCGGGTTCCAAGGGTGAACAGGGTTTCATG
GGTCCACCGGGCCCACAAGGTCAGCCGGGTCTGCCTGGTTTCCCG
GGCTTCCCGGGTGCCAAAGGCGACAAAGGTAGCAAGGGCGAAGTT
GGCTTTCCGGGTCTGGCGGGTTCGCCGGGCATTCCGGGCTCGAAG
GGCGAGCAGGGTTTCATGGGCCCACCGGGTCCGCAGGGTCAGCCT
GGCCTGCCGGGATTCCCAGGTTTTCCGGGAGCGAAAGGCGACAAG
GGTAGTAAGGGTGAGGTCGGTTTTCCAGGCTTGGCGGGCTCTCCC
GGTATCCCGGGCTCTAAGGGCGAGCAAGGCTTTATGGGTCCACCG
GGTCCGCAAGGTCAACCTGGATTACCGGGATTCCCAGGCTTTCCG
GGCGCGAAAGGCGATAAAGGCAGCAAGGGTGAGGTGGGCTTCCCG
GGCCTCGCGGGTAGCCCGGGCATCCCGGGTAGCAAGGGTGAGCAG
GGCTTCATGGGTCCTCCGGGTCCGCAGGGCCAACCGGGCCTGCCG
GGATTCCCGGGTTTCCCGGGCGCTAAAGGCGACAAAGGCAGCAAG
GGTGAGGTTGGTTTTCCGGGTCTGGCAGGTAGCCCGGGCATTCCG
GGCTCCAAGGGCGAACAGGGTTTTATGGGTCCACCGGGCCCTCAA
GGTCAGCCGGGCCTGCCG

4) C4P7Cb amino acid sequence (the amino acid sequence of the repeating unit is Gfpgfpgakgdkgskgevgfpglagspgipgskgeqgfmgppgpq, SEQ ID NO: 10, and the number of repeating units is 8; the amino acid sequence of C4P7Cb is SEQ ID NO: 11):

(SEQ ID NO: 11)
Gfpgfpgakgdkgskgevgfpglagspgipgskgeqgfmgppgpq
Gfpgfpgakgdkgskgevgfpglagspgipgskgeqgfmgppgpq
Gfpgfpgakgdkgskgevgfpglagspgipgskgeqgfmgppgpq
Gfpgfpgakgdkgskgevgfpglagspgipgskgeqgfmgppgpq
Gfpgfpgakgdkgskgevgfpglagspgipgskgeqgfmgppgpq
Gfpgfpgakgdkgskgevgfpglagspgipgskgeqgfmgppgpq
Gfpgfpgakgdkgskgevgfpglagspgipgskgeqgfmgppgpq
Gfpgfpgakgdkgskgevgfpglagspgipgskgeqgfmgppgpq
C4P7Cb base sequence (SEQ ID NO: 12):
(SEQ ID NO: 12)
GGATTTCCCGGGTTTCCGGGTGCGAAAGGCGACAAGGGTTCCAAG
GGTGAAGTTGGTTTTCCGGGGCTCGCTGGTAGCCCGGGCATCCCG
GGTAGCAAGGGCGAACAGGGTTTCATGGGCCCACCGGGCCCGCAG
GGTTTTCCGGGTTTCCCGGGCGCGAAAGGCGACAAAGGCAGCAAG
GGTGAAGTGGGCTTTCCGGGCCTGGCGGGTAGTCCGGGCATCCCG
GGCTCGAAGGGCGAACAGGGCTTTATGGGCCCACCAGGCCCACAG
GGCTTCCCGGGATTCCCGGGTGCCAAAGGCGATAAAGGTTCAAAG
GGCGAAGTCGGTTTCCCGGGTCTGGCAGGCTCTCCGGGCATCCCG
GGCTCTAAGGGTGAACAAGGTTTCATGGGTCCACCGGGCCCGCAG
GGCTTTCCGGGTTTTCCGGGTGCGAAAGGCGACAAGGGCAGCAAA
GGTGAGGTTGGCTTTCCGGGCTTGGCGGGTAGCCCGGGTATCCCG
GGTAGCAAGGGTGAGCAGGGTTTCATGGGTCCGCCTGGACCGCAA
GGTTTCCCGGGTTTCCCGGGTGCGAAAGGTGATAAAGGTAGCAAG
GGCGAGGTAGGTTTTCCGGGTCTGGCAGGCTCCCCGGGCATTCCG
GGCTCTAAGGGTGAGCAGGGTTTCATGGGTCCGCCAGGTCCGCAA
GGTTTCCCGGGATTTCCGGGTGCTAAAGGTGATAAAGGTTCCAAA
GGTGAAGTGGGTTTTCCAGGCCTGGCCGGCAGCCCGGGCATTCCG
GGCTCCAAGGGCGAGCAGGGCTTCATGGGTCCGCCTGGCCCTCAA
GGCTTCCCCGGCTTCCCGGGAGCTAAGGGCGACAAAGGTTCCAAG
GGCGAGGTGGGCTTCCCGGGCTTGGCGGGTAGCCCGGGCATTCCG
GGTAGCAAGGGTGAGCAAGGCTTTATGGGTCCGCCAGGTCCGCAA
GGGTTCCCGGGATTCCCGGGTGCGAAAGGTGATAAAGGCTCGAAG
GGTGAGGTTGGTTTTCCGGGTCTGGCAGGTAGCCCGGGGATTCCG
GGTAGCAAAGGCGAACAAGGTTTCATGGGTCCTCCGGGTCCACAG

5) C4P7Cc amino acid sequence (the amino acid sequence of the repeating unit is Gfpgfpgakgdkgskgevgfpglagspgipgskgeqgfm, SEQ ID NO: 13, and the number of repeating units is 8; the amino acid sequence of C4P7Cc is SEQ ID NO: 14):

(SEQ ID NO: 14)

Gfpgfpgakgdkgskgevgfpglagspgipgskgeqgfm
Gfpgfpgakgdkgskgevgfpglagspgipgskgeqgfm
Gfpgfpgakgdkgskgevgfpglagspgipgskgeqgfm
Gfpgfpgakgdkgskgevgfpglagspgipgskgeqgfm
Gfpgfpgakgdkgskgevgfpglagspgipgskgeqgfm
Gfpgfpgakgdkgskgevgfpglagspgipgskgeqgfm
Gfpgfpgakgdkgskgevgfpglagspgipgskgeqgfm
C4P7Cc base sequence (SEQ ID NO: 15):
(SEQ ID NO: 15)
GGATTTCCCGGGTTCCCGGGTGCGAAAGGTGATAAAGGCAGCAAG
GGTGAAGTCGGTTTTCCGGGTCTGGCAGGCAGCCCGGGTATCCCG
GGTAGCAAAGGCGAACAGGGCTTTATGGGTTTCCCGGGCTTCCCA
GGTGCGAAGGGCGATAAAGGTTCGAAAGGTGAGGTAGGTTTCCCG
GGTTTAGCAGGTTCCCCGGGCATTCCGGGCAGCAAGGGTGAACAG
GGTTTCATGGGCTTTCCGGGCTTCCCAGGAGCTAAAGGCGACAAA
GGTTCTAAGGGTGAAGTGGGCTTCCCGGGTCTGGCTGGTAGCCCG
GGCATCCCGGGCTCCAAGGGTGAGCAGGGTTTCATGGGTTTTCCG
GGCTTCCCAGGCGCGAAAGGCGACAAAGGCAGCAAGGGCGAGGTG
GGTTTTCCGGGTTTGGCGGGTAGCCCGGGTATTCCGGGTTCGAAG
GGTGAACAAGGTTTCATGGGTTTTCCGGGATTCCCAGGCGCGAAA
GGCGATAAGGGCAGCAAGGGCGAGGTTGGCTTCCCGGGACTGGCC
GGAAGCCCGGGTATCCCGGGATCTAAGGGCGAACAAGGCTTTATG
GGTTTCCCGGGTTTTCCTGGTGCGAAAGGCGATAAAGGCTCCAAG
GGCGAGGTTGGTTTTCCAGGCCTGGCTGGCTCTCCGGGCATTCCG
GGTAGTAAGGGTGAGCAGGGTTTTATGGGTTTTCCGGGCTTCCCG
GGTGCAAAGGGTGACAAAGGTAGCAAGGGTGAAGTTGGCTTTCCG
GGTCTGGCGGGTTCCCCGGGCATTCCGGGTAGCAAAGGTGAGCAA
GGTTTTATGGGTTTTCCGGGCTTCCCGGGTGCCAAAGGCGACAAA
GGTAGCAAGGGAGAGGTGGGCTTCCCGGGATTGGCGGGTTCCCCG
GGCATCCCGGGCTCAAAGGGTGAACAGGGTTTCATG

6) C4P7Cd amino acid sequence (the amino acid sequence of the repeating unit is Gfpgfpgakgdkgskgevgfpglagspgipgskgeq, SEQ ID NO: 16, and the number of repeating units is 10; the amino acid sequence of C4P7Cd is SEQ ID NO: 17):

(SEQ ID NO: 17)
Gfpgfpgakgdkgskgevgfpglagspgipgskgeq
Gfpgfpgakgdkgskgevgfpglagspgipgskgeq
Gfpgfpgakgdkgskgevgfpglagspgipgskgeq
Gfpgfpgakgdkgskgevgfpglagspgipgskgeq
Gfpgfpgakgdkgskgevgfpglagspgipgskgeq
Gfpgfpgakgdkgskgevgfpglagspgipgskgeq
Gfpgfpgakgdkgskgevgfpglagspgipgskgeq
Gfpgfpgakgdkgskgevgfpglagspgipgskgeq
Gfpgfpgakgdkgskgevgfpglagspgipgskgeq
Gfpgfpgakgdkgskgevgfpglagspgipgskgeq
C4P7Cd base sequence (SEQ ID NO: 18):
(SEQ ID NO: 18)
GGATTTCCCGGGTTCCCAGGTGCCAAGGGTGATAAAGGCAGCAAG
GGCGAGGTGGGCTTCCCGGGCCTGGCTGGTTCACCGGGCATTCCG
GGCAGTAAGGGTGAACAGGGTTTTCCGGGTTTCCCCGGCGCTAAA
GGCGACAAGGGTAGCAAGGGGGAAGTAGGCTTCCCGGGTTTAGCT
GGCTCTCCGGGCATTCCGGGCTCCAAGGGCGAACAGGGTTTCCCG
GGCTTTCCGGGAGCGAAAGGCGACAAAGGCAGCAAGGGCGAAGTT
GGTTTCCCGGGTCTGGCAGGTTCTCCGGGTATCCCGGGTAGCAAA
GGCGAGCAGGGTTTCCCCGGCTTCCCGGGTGCGAAAGGTGATAAA
GGTAGCAAGGGTGAGGTGGGTTTCCCTGGCCTGGCCGGTTCTCCG
GGCATCCCGGGTTCTAAGGGAGAACAAGGTTTTCCGGGTTTTCCG
GGTGCGAAAGGCGACAAAGGGTCCAAGGGTGAGGTTGGTTTTCCG
GGTCTGGCGGGTTCACCGGGCATCCCGGGTAGCAAGGGCGAACAA
GGCTTTCCAGGCTTCCCGGGTGCGAAAGGTGACAAAGGTTCCAAG
GGCGAAGTTGGTTTTCCGGGTTTGGCGGGTAGCCCGGGTATCCCG
GGTTCCAAGGGTGAGCAAGGTTTTCCGGGATTCCCGGGTGCCAAA
GGTGACAAAGGTTCGAAGGGTGAAGTGGGTTTTCCAGGCTTGGCG
GGTAGCCCGGGTATCCCGGGTTCCAAGGGTGAGCAGGGCTTCCCG
GGTTTCCCGGGTGCAAAAGGTGATAAAGGCTCCAAAGGTGAGGTG
GGCTTCCCTGGTCTCGCGGGTAGCCCGGGCATCCCGGGTAGCAAA
GGTGAGCAGGGTTTCCCGGGCTTTCCAGGAGCTAAAGGGGATAAA
GGCAGCAAGGGCGAGGTTGGCTTCCCGGGTCTGGCGGGTTCGCCG
GGCATTCCGGGCAGCAAAGGCGAACAGGGCTTTCCGGGTTTTCCG
GGTGCAAAGGGTGATAAAGGTAGCAAGGGCGAGGTCGGTTTTCCG
GGCCTGGCAGGTAGCCCAGGCATTCCGGGTTCGAAGGGCGAACAA

7) C4P7Ce amino acid sequence (the amino acid sequence of the repeating unit is Gakgdkgskgevgfpglagspgipgskgeqgfmgppgpqgqpglp, SEQ ID NO: 19, and the number of repeating units is 8; the amino acid sequence of C4P7Ce is SEQ ID NO: 20):

(SEQ ID NO: 20)

Gakgdkgskgevgfpglagspgipgskgeqgfmgppgpqgqpglp
Gakgdkgskgevgfpglagspgipgskgeqgfmgppgpqgqpglp
Gakgdkgskgevgfpglagspgipgskgeqgfmgppgpqgqpglp
Gakgdkgskgevgfpglagspgipgskgeqgfmgppgpqgqpglp
Gakgdkgskgevgfpglagspgipgskgeqgfmgppgpqgqpglp
Gakgdkgskgevgfpglagspgipgskgeqgfmgppgpqgqpglp
Gakgdkgskgevgfpglagspgipgskgeqgfmgppgpqgqpglp
C4P7Ce base sequence (SEQ ID NO: 21):
(SEQ ID NO: 21)
GGGGCTAAAGGAGACAAGGGCAGCAAGGGCGAGGTTGGTTTCCCG
GGCTTGGCGGGTTCTCCGGGTATCCCGGGCTCCAAGGGTGAACAG
GGTTTTATGGGTCCGCCAGGTCCGCAGGGCCAACCTGGTCTGCCG
GGCGCGAAAGGTGACAAAGGTAGCAAGGGCGAAGTTGGTTTTCCG
GGCCTGGCCGGCAGCCCGGGCATTCCGGGCTCCAAAGGCGAGCAA
GGCTTCATGGGTCCGCCTGGCCCACAGGGTCAACCGGGTCTGCCG
GGCGCAAAAGGCGATAAAGGCAGCAAGGGCGAAGTAGGTTTCCCG
GGCCTTGCTGGCTCTCCGGGTATTCCGGGTAGCAAGGGTGAACAG
GGTTTTATGGGCCCACCGGGACCACAGGGTCAACCGGGTCTGCCG
GGTGCTAAAGGTGATAAAGGCAGCAAGGGCGAGGTTGGTTTTCCG
GGATTGGCGGGTTCTCCGGGCATCCCGGGTTCCAAGGGCGAACAA
GGTTTCATGGGACCGCCTGGCCCACAGGGTCAACCGGGTCTGCCT
GGTGCAAAAGGCGACAAAGGTAGCAAAGGCGAGGTCGGTTTTCCG
GGCCTCGCGGGTAGCCCGGGAATCCCGGGTAGTAAGGGCGAACAG
GGTTTCATGGGTCCACCGGGCCCACAGGGTCAGCCGGGTTTACCG
GGTGCCAAAGGCGATAAAGGGTCGAAGGGCGAGGTGGGCTTCCCG
GGTCTGGCAGGCAGCCCGGGTATTCCGGGCTCCAAGGGTGAGCAG
GGTTTTATGGGTCCACCGGGACCGCAAGGTCAACCGGGTOTGCCG
GGCGCTAAAGGCGATAAGGGTAGCAAAGGCGAGGTGGGCTTCCCG
GGTCTGGCGGGTAGCCCGGGCATTCCGGGTTCCAAGGGTGAGCAG
GGTTTCATGGGTCCGCCTGGTCCTCAAGGCCAGCCGGGGCTGCCC
GGCGCGAAAGGTGACAAAGGTAGCAAGGGCGAAGTGGGTTTTCCG
GGTTTGGCGGGTTCACCGGGCATCCCGGGCTCGAAGGGTGAACAG
GGTTTCATGGGTCCTCCGGGCCCGCAGGGACAACCGGGACTGCCG

8) C4P7Cg amino acid sequence (the amino acid sequence of the repeating unit is Gakgdkgskgevgfpglagspgipgskgeqgfm, SEQ ID NO: 22, and the number of repeating units is 10; the amino acid sequence of C4P7Cg is SEQ ID NO: 23):

(SEQ ID NO: 23)
Gakgdkgskgevgfpglagspgipgskgeqgfm
Gakgdkgskgevgfpglagspgipgskgeqgfm
Gakgdkgskgevgfpglagspgipgskgeqgfm
Gakgdkgskgevgfpglagspgipgskgeqgfm
Gakgdkgskgevgfpglagspgipgskgeqgfm
Gakgdkgskgevgfpglagspgipgskgeqgfm
Gakgdkgskgevgfpglagspgipgskgeqgfm
Gakgdkgskgevgfpglagspgipgskgeqgfm
Gakgdkgskgevgfpglagspgipgskgeqgfm
Gakgdkgskgevgfpglagspgipgskgeqgfm
C4P7Cg base sequence (SEQ ID NO: 24):
(SEQ ID NO: 24)
GGGGCTAAAGGAGACAAAGGTTCGAAAGGCGAGGTGGGCTTCCCA
GGTCTGGCCGGTTCCCCGGGCATTCCGGGTAGCAAAGGCGAACAA
GGTTTCATGGGTGCTAAAGGCGATAAAGGTAGCAAGGGTGAGGTT
GGCTTCCCAGGCCTGGCTGGTTCGCCGGGCATTCCGGGCTCTAAG
GGTGAACAAGGTTTCATGGGTGCAAAAGGTGATAAGGGTAGCAAG
GGAGAAGTCGGTTTTCCGGGATTGGCGGGTAGCCCGGGTATCCCG
GGCAGCAAGGGCGAGCAGGGTTTTATGGGTGCAAAGGGCGACAAA
GGTAGCAAGGGTGAGGTGGGCTTTCCGGGCCTCGCGGGTAGCCCT
GGCATCCCGGGTTCCAAAGGTGAGCAAGGCTTCATGGGTGCTAAA
GGTGATAAAGGCTCCAAAGGTGAAGTGGGTTTTCCGGGCCTGGGG
GTAGCCCGGGCATTCCGGGAAGCAAGGGCGAACAGGGTTTTATGG
GCGCGAAGGGTGATAAAGGTAGTAAGGGCGAAGTTGGTTTCCCGG
GCCTGGCTGGCTCTCCGGGTATCCCGGGCTCCAAAGGCGAGCAGG
GTTTCATGGGTGCGAAAGGTGACAAGGGTAGCAAGGGTGAGGTGG
GTTTCCCAGGTTTGGCGGGTAGCCCGGGCATTCCGGGTAGCAAGG
GTGAACAAGGTTTCATGGGTGCGAAAGGTGACAAAGGCAGCAAGG
GCGAGGTTGGTTTCCCGGGTCTGGCGGGTAGCCCGGGCATCCCGG
GCTCTAAGGGCGAGCAGGGTTTTATGGGTGCCAAAGGCGACAAGG
GCTCAAAGGGTGAAGTCGGTTTTCCGGGTTTAGCCGGTTCCCCGG
GCATCCCGGGTTCTAAGGGTGAACAGGGCTTCATGGGCGCGAAAG
GAGATAAAGGCAGCAAAGGGGAAGTTGGTTTTCCAGGCCTGGCAG
GCTCGCCGGGTATCCCGGGTTCCAAGGGCGAGCAGGGTTTTATG

9) C4P7Ea amino acid sequence (the amino acid sequence of the repeating unit is Glpgtpgptgpagqkgepgsdgipgsagekgepglp, SEQ ID NO: 25, and the number of repeating units is 10; the amino acid sequence of C4P7Ea is SEQ ID NO: 26):

(SEQ ID NO: 26)
Glpgtpgptgpagqkgepgsdgipgsagekgepglp
Glpgtpgptgpagqkgepgsdgipgsagekgepglp
Glpgtpgptgpagqkgepgsdgipgsagekgepglp
Glpgtpgptgpagqkgepgsdgipgsagekgepglp
Glpgtpgptgpagqkgepgsdgipgsagekgepglp
Glpgtpgptgpagqkgepgsdgipgsagekgepglp
Glpgtpgptgpagqkgepgsdgipgsagekgepglp
Glpgtpgptgpagqkgepgsdgipgsagekgepglp
Glpgtpgptgpagqkgepgsdgipgsagekgepglp
Glpgtpgptgpagqkgepgsdgipgsagekgepglp
C4P7E a base sequence (SEQ ID NO: 27):
(SEQ ID NO: 27)
GGACTACCCGGGACTCCGGGCCCAACCGGCCCAGCAGGCCAAAAG
GGTGAACCGGGGTCCGACGGCATTCCGGGCAGCGCAGGTGAGAAA
GGCGAACCGGGCTTGCCGGGGTTGCCGGGAACCCCGGGTCCGACC
GGTCCAGCTGGCCAGAAAGGTGAGCCAGGAAGCGATGGCATCCCG
GGCTCGGCCGGTGAAAAAGGCGAGCCGGGTCTCCCGGGTCTGCCG
GGAACCCCGGGTCCGACAGGCCCGGCTGGTCAGAAGGGCGAGCCG
GGTAGCGACGGCATCCCGGGCAGCGCTGGTGAAAAAGGTGAGCCC
GGTCTGCCGGGCTTGCCCGGCACCCCGGGACCGACGGGCCCAGCA
GGCCAGAAGGGCGAACCGGGTTCGGATGGTATTCCGGGCTCTGCC
GGTGAGAAAGGCGAGCCCGGCTTGCCAGGCCTGCCTGGCACCCCG
GGTCCGACCGGTCCGGGGGGCCAAAAAGGCGAGCCGGGTAGCGAT
GGTATCCCGGGCTCAGCCGGTGAGAAGGGTGAGCCGGGCCTGCCG
GGCCTGCCTGGTACGCCGGGTCCGACCGGTCCGGCGGGCCAAAAG
GGTGAACCGGGCTCCGACGGCATTCCGGGTTCTGCGGGTGAGAAA
GGTGAACCGGGCCTGCCGGGCCTGCCCGGCACCCCGGGTCCTACG
GGTCCGGCTGGTCAGAAAGGCGAGCCGGGCTCCGATGGCATTCCG
GGTTCTGCGGGTGAGAAGGGTGAACCGGGCTTGCCAGGTCTGCCG
GGCACCCCGGGTCCGACGGGTCCGGCGGGTCAGAAGGGTGAGCCG
GGTTCCGATGGTATCCCGGGCAGCGCGGGAGAAAAAGGTGAACCG
GGTCTGCCGGGTCTTCCGGGTACTCCGGGTCCGACCGGCCCTGCG
GGTCAGAAGGGTGAGCCGGGAAGCGACGGCATCCCGGGCAGCGCG
GGGGAGAAGGGTGAACCGGGTTTACCTGGCCTGCCGGGAACCCCG
GGCCCTACCGGTCCGGCGGGTCAAAAGGGCGAACCGGGCAGCGAC
GGTATCCCGGGTAGCGCAGGCGAAAAAGGTGAACCGGGCCTGCCG

10) C4P7Eb amino acid sequence (the amino acid sequence of the repeating unit is Gptgpagqkgepgsdgipgsagekgepglp, SEQ ID NO: 28, and the number of repeating units is 10; the amino acid sequence of C4P7Eb is SEQ ID NO: 29):

(SEQ ID NO: 29)
Gptgpagqkgepgsdgipgsagekgepglp
Gptgpagqkgepgsdgipgsagekgepglp
Gptgpagqkgepgsdgipgsagekgepglp
Gptgpagqkgepgsdgipgsagekgepglp
Gptgpagqkgepgsdgipgsagekgepglp
Gptgpagqkgepgsdgipgsagekgepglp
Gptgpagqkgepgsdgipgsagekgepglp
Gptgpagqkgepgsdgipgsagekgepglp
Gptgpagqkgepgsdgipgsagekgepglp
Gptgpagqkgepgsdgipgsagekgepglp
C4P7Eb base sequence (SEQ ID NO: 30):
(SEQ ID NO: 30)
GGTCCCACAGGACCGGCAGGCCAGAAAGGTGAGCCGGGTTCCGAC
GGCATCCCGGGTTCGGCGGGTGAGAAAGGCGAGCCGGGTTTACCG
GGTCCGACCGGTCCCGCGGGTCAAAAGGGCGAGCCGGGTAGCGAT
GGCATTCCGGGTTCTGCGGGTGAAAAGGGCGAACCGGGCCTCCCG
GGTCCTACCGGTCCGGCGGGTCAGAAAGGCGAACCGGGCAGCGAT
GGCATCCCGGGCAGCGCGGGCGAGAAAGGCGAACCGGGCCTGCCG
GGCCCGACCGGACCAGCTGGGCAAAAAGGTGAACCGGGCAGCGAC
GGCATCCCGGGTTCTGCAGGCGAGAAAGGTGAACCAGGCCTGCCG
GGACCGACCGGTCCGGCAGGCCAGAAAGGTGAGCCTGGCAGTGAT
GGTATTCCGGGTTCTGCCGGTGAAAAAGGTGAGCCGGGCCTGCCG
GGGCCAACGGGCCCAGCCGGACAAAAAGGTGAGCCGGGTTCCGAC
GGCATCCCGGGCTCCGCCGGTGAAAAGGGTGAGCCGGGCCTGCCT
GGCCCAACGGGTCCGGCTGGCCAAAAGGGCGAGCCGGGTAGCGAC
GGCATTCCGGGCAGCGCGGGTGAGAAGGGTGAGCCGGGATTGCCG
GGTCCGACTGGTCCTGCGGGCCAGAAGGGTGAACCGGGTTCCGAC
GGCATCCCCGGCTCGGCGGGTGAAAAGGGCGAACCGGGTCTGCCT
GGTCCGACCGGCCCAGCGGGTCAGAAGGGTGAACCGGGTAGCGAT
GGAATCCCGGGTAGCGCTGGTGAAAAGGGCGAGCCGGGCCTGCCG
GGTCCGACCGGTCCGGCAGGCCAGAAGGGTGAACCGGGTAGCGAT
GGTATTCCGGGTAGCGCGGGCGAAAAAGGTGAGCCGGGCTTGCCG

11) C4P7Ec amino acid sequence (the amino acid sequence of the repeating unit is Gfpgfpgakgdkgskgevgfpglagspgipgsk, SEQ ID NO: 31, and the number of repeating units is 10; the amino acid sequence of C4P7Ec is SEQ ID NO: 32):

(SEQ ID NO: 32)
Gfpgfpgakgdkgskgevgfpglagspgipgsk
Gfpgfpgakgdkgskgevgfpglagspgipgsk
Gfpgfpgakgdkgskgevgfpglagspgipgsk
Gfpgfpgakgdkgskgevgfpglagspgipgsk
Gfpgfpgakgdkgskgevgfpglagspgipgsk
Gfpgfpgakgdkgskgevgfpglagspgipgsk
Gfpgfpgakgdkgskgevgfpglagspgipgsk
Gfpgfpgakgdkgskgevgfpglagspgipgsk
Gfpgfpgakgdkgskgevgfpglagspgipgsk
C4P7Ec base sequence (SEQ ID NO: 33):
(SEQ ID NO: 33)
GGATTTCCCGGGTTCCCAGGCGCAAAAGGTGATAAAGGCAGCAAG
GGCGAGGTTGGTTTTCCAGGTTTAGCTGGTAGCCCGGGTATCCCG
GGTAGCAAGGGCTTCCCGGGTTTTCCGGGTGCTAAAGGCGACAAA
GGCTCCAAGGGCGAAGTCGGTTTCCCGGGTTTGGCGGGTAGCCCG
GGTATCCCGGGTAGTAAGGGCTTTCCGGGATTCCCAGGCGCGAAA
GGTGACAAAGGTAGCAAGGGCGAAGTTGGCTTCCCGGGTTTGGCG
GGTTCCCCGGGTATCCCGGGGTCCAAGGGCTTCCCCGGATTCCCG
GGCGCGAAAGGCGATAAAGGTAGCAAGGGTGAAGTGGGTTTTCCG
GGTCTCGCTGGCAGCCCGGGTATTCCGGGCTCCAAGGGCTTTCCA
GGCTTTCCGGGTGCGAAAGGCGATAAAGGTAGCAAGGGTGAGGTG
GGTTTTCCGGGTCTGGCAGGTAGCCCTGGCATCCCGGGCTCGAAG
GGGTTCCCGGGCTTCCCGGGAGCCAAGGGTGATAAAGGTTCTAAG
GGTGAGGTCGGTTTTCCGGGCCTGGCCGGTAGCCCTGGTATCCCG
GGGAGCAAGGGTTTCCCGGGTTTTCCGGGTGCCAAAGGCGATAAA
GGCTCTAAGGGCGAGGTGGGCTTCCCCGGTCTGGCGGGTAGCCCG
GGTATTCCGGGTTCTAAGGGCTTCCCGGGTTTTCCGGGTGCGAAA
GGTGACAAGGGCTCCAAGGGTGAAGTTGGTTTTCCGGGTCTGGCT
GGTAGCCCGGGTATCCCGGGTAGCAAGGGCTTCCCGGGTTTCCCG
GGCGCGAAAGGCGACAAAGGTTCAAAGGGTGAAGTTGGTTTTCCT
GGCCTGGCAGGCAGCCCGGGCATTCCGGGTTCCAAAGGTTTTCCG
GGCTTCCCGGGTGCGAAAGGTGACAAAGGCTCGAAGGGTGAGGTG
GGCTTCCCGGGTCTGGCAGGTTCTCCTGGCATTCCGGGTTCGAAA

Each of the aforementioned encoding nucleotide sequences was commercially synthesized. Subsequently, each of the above encoding nucleotide sequences (a collagen-processing enzyme cleavage site with the amino acid sequence of ENLY FQ (SEQ ID NO: 56) and the nucleotide sequence of GAAAACCTGTATTTCCAG (SEQ ID NO: 57) was added at the 5′ end) was inserted between the K pnl and X hol cleavage sites of the pET-28a-Trx-His expression vector to generate a recombinant expression plasmid.

3. The successfully constructed expression plasmid was transformed into E. coli competent cell BL21 (DE3). Specifically, the process was as follows: (1) taking out E. coli competent cells BL21 (DE3) from an ultra-low temperature refrigerator and placing them on ice. Once the cells were half-thawed, pipette 2 μl of the plasmid to be transformed into the E. coli competent cells BL21 (DE3) and gently mix 2-3 times. (2) Placing the mixture on ice in an ice bath for 30 minutes, then heat shocking in a water bath at 42° C. for 45-90 s, and removing the mixture and placing it on ice in an ice bath for 2 minutes. (3) Transferring the mixture to a biosafety cabinet, adding 700 μl of liquid LB medium, and then incubating at 37°° C. and at 220 rpm for 60 min. (4) Taking 200 μl of the bacterial solution and evenly spreading it on a LB plate containing ampicillin sodium. (5) Incubating the plate in an incubator at 37° C. for 15-17 h, until colonies of uniform size had grown.

4. 5-6 single colonies were picked from the LB plate with transformed cells and placed in shake flasks containing LB medium supplemented with antibiotic stock solution, followed by incubation in a shaker at 220 rpm and a constant temperature of 37° C. for 7 hours. The cultured shake flask was then cooled to 16° C., and IPTG was added to induce expression for a specific period. The bacterial solution was dispensed into centrifuge bottles and centrifuged at 8000 rpm and 4° C. for 10 minutes. The bacterial cells were collected, the bacterial cell weight was recorded, and samples (labeled “bacterial solution”) were taken for electrophoresis detection.

5. The collected bacterial cells were resuspended in an equilibrium working solution (200 mM sodium chloride, 25 mM Tris, 20 mM imidazole at pH 8.0), and the bacterial solution was cooled to ≤15° C. The solution was homogenized by two rounds of high-pressure homogenization (samples were taken after each round and labeled “homogenization 1” and “homogenization 2”, respectively), and the bacterial solution was collected after homogenization. The homogenized bacterial solution was divided into centrifuge bottles and centrifuged at 17,000 rpm and 4° C. for 30 minutes. The supernatant was collected, and both the supernatant (labeled “Supernatant”) and the pellets were taken for electrophoresis detection.

6. Purifying and enzymatically digesting the recombinant type IV humanized collagen. Specifically, the processes were as follows. (1) Crude purification: a. Washing the column material (Ni6FF, Cytiva) with water for 5 CVs. b. Equilibrating the column material with equilibrium solution (200 mM sodium chloride, 25 mM Tris, 20 mM imidazole, at pH 8.0) for 5 CVs. c. Loading: loading the obtained after centrifugation onto the column material until the liquid flowed through the column material completely, and then taking the flow through (labeled “Flow through”) for electrophoresis detection. d. Washing impurity proteins: Adding 25 mL of washing solution (200 mM sodium chloride, 25 mM Tris, 20 mM imidazole) until the liquid flowed through completely, and taking the impurity-washing flow through (labeled “Impurity-washing”) for electrophoresis detection. e. Collecting the target protein: adding 20 mL of eluate (200 mM sodium chloride, 25 mM Tris, 250 mM imidazole, at pH 8.0), and collecting the flow-through liquid (marked: elution), detecting the protein concentration and calculating the protein amount, and performing electrophoresis detection. f. Washing the column material with 1 M imidazole working solution (labeled “Washing with 1 M”). g. Washing the column material with purified water. (2) Enzymatically digesting: according to the ratio of total protein amount to total TEV enzyme amount of 50:1, adding TEV enzyme for digestion at 16° C. for 4 h, and sampling for electrophoresis detection (labeled “After digestion”). Putting the enzymatically digested protein solution into a dialysis bag and dialyzing at 4°° C. for 2 h, then transferring it to a fresh dialysate for dialysis at 4° C. overnight (labeled “Liquid exchange”).

(3) Fine purification: a. equilibrating the column material (Capto Q, Cytiva): equilibrating the column material using solution A (20 mM Tris, 20 mM sodium chloride, at pH 8.0) at a flow rate of 10 ml/min. b. Loading: loading the sample at a flow rate of 5 ml/min and collecting the flow through (labeled “QFL”), and performing electrophoresis detection. c. Gradient eluting: Setting 0-15% solution B (20 mM Tris, 1 M sodium chloride, at pH 8.0) for 2 min followed by holding for 3 CVs, 15-30% solution B for 2 min followed by holding for 3 CVs, 30-50% solution B for 2 min followed by holding 3 CV s, respectively, collecting the peak and performing electrophoresis detection (labeled “Washing with solution B”). d. Washing the column material. The protein was stored at 4° C.

### 7. Electrophoretic Detection

Specifically, the processes were as follows. 40 μL of sample solution was taken, and 10 μL of 5× protein loading buffer (250 mM Tris-HCl at pH 6.8, 10% SDS, 0.5% bromophenol blue, 50% glycerol, 5% β-mercaptoethanol) was added. The mixture was heated in boiling water at 100° C. for 10 minutes, then 10 μL was loaded into each well of an SDS-PAGE protein gel. The gel was run at 80 V for 2 hours, stained for 20 minutes using Coomassie brilliant blue staining solution (0.1% Coomassie brilliant blue R-250, 25% isopropanol, 10% glacial acetic acid), and then decolorized using protein decolorization solution (10% acetic acid, 5% ethanol).

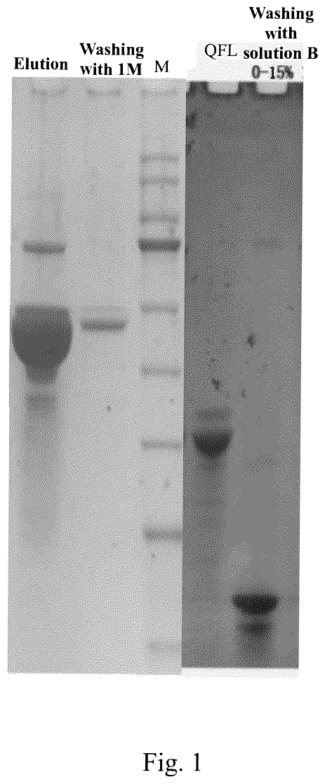

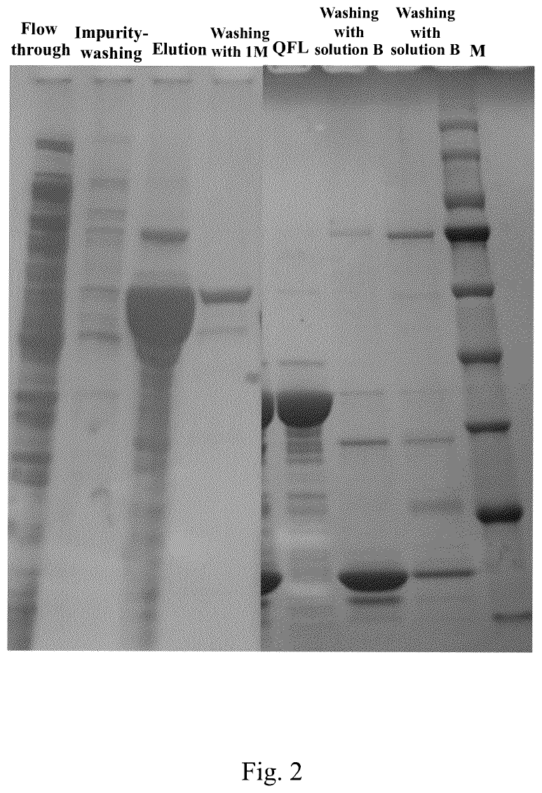

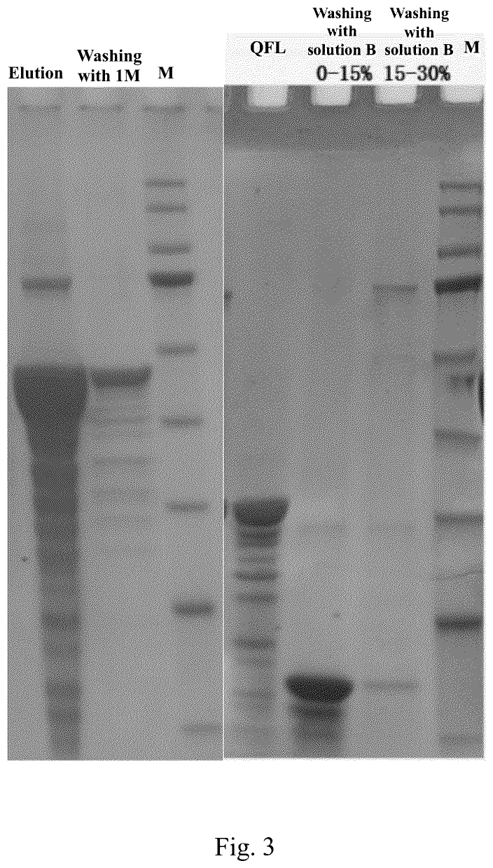

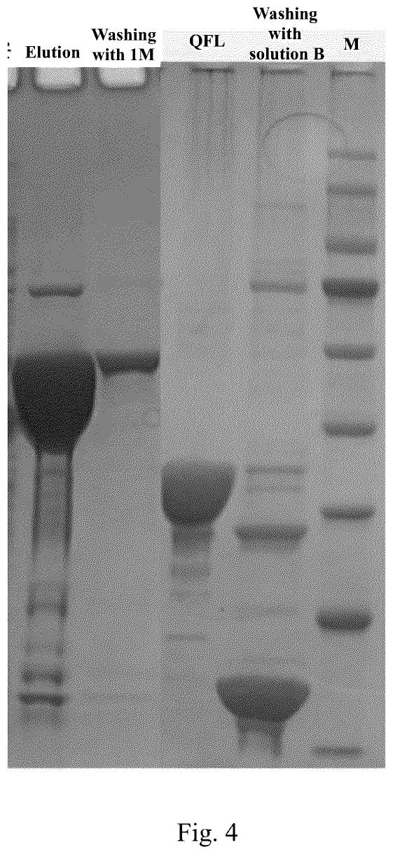

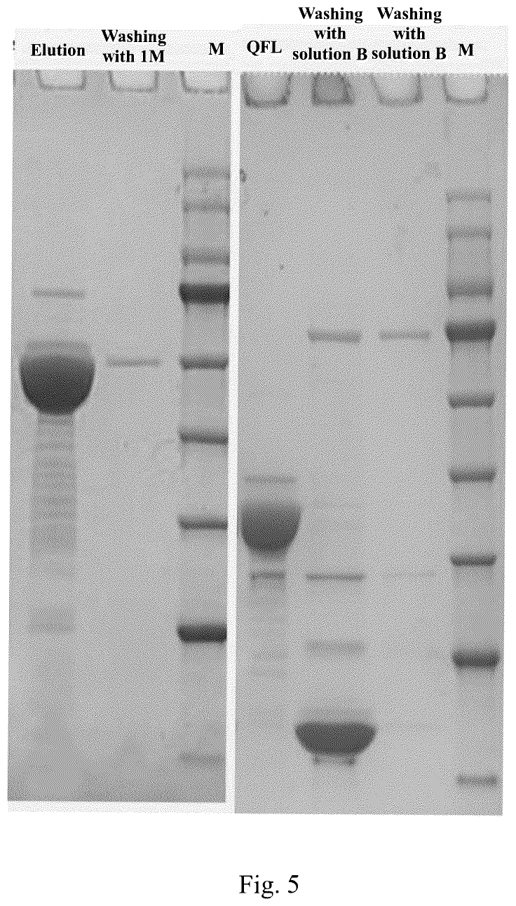

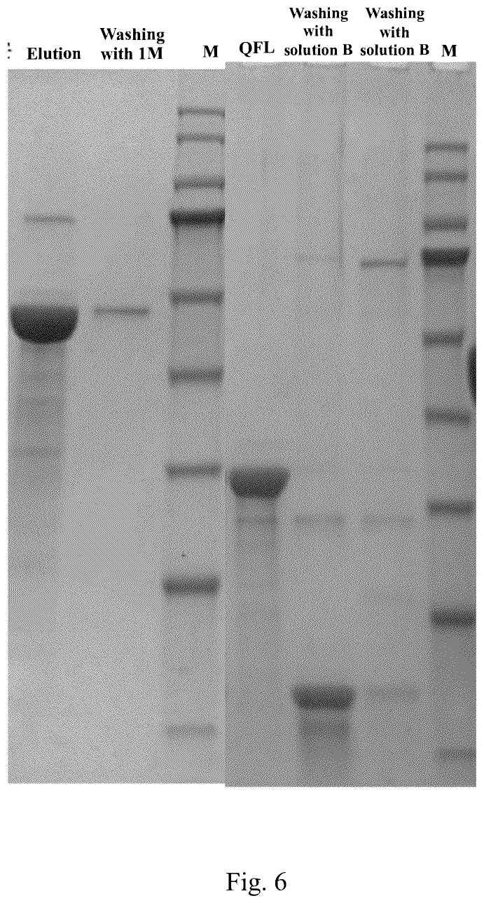

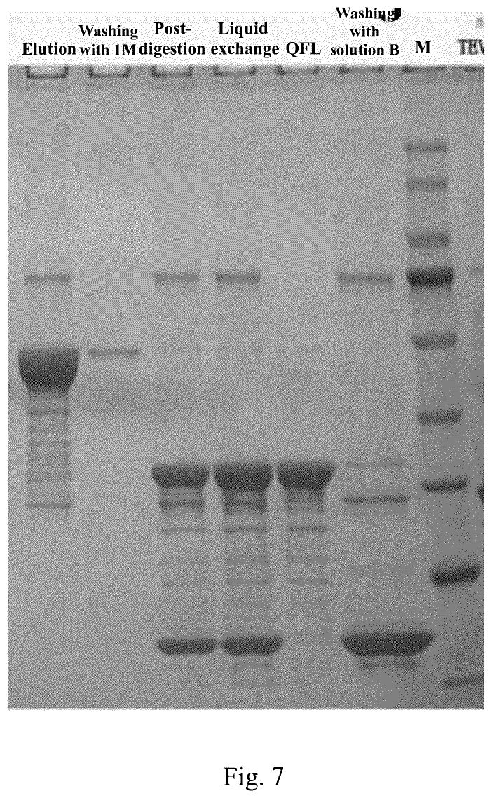

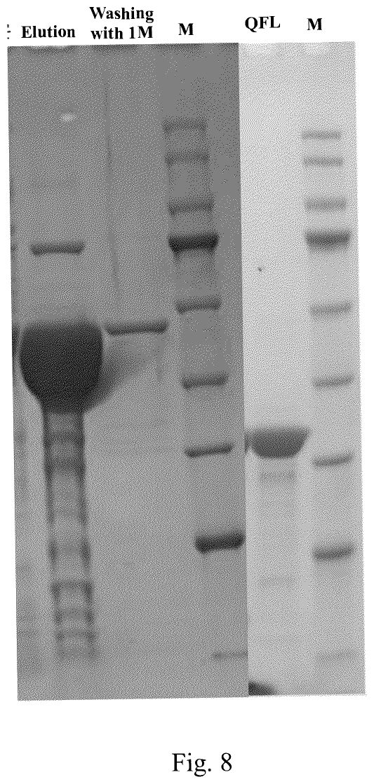

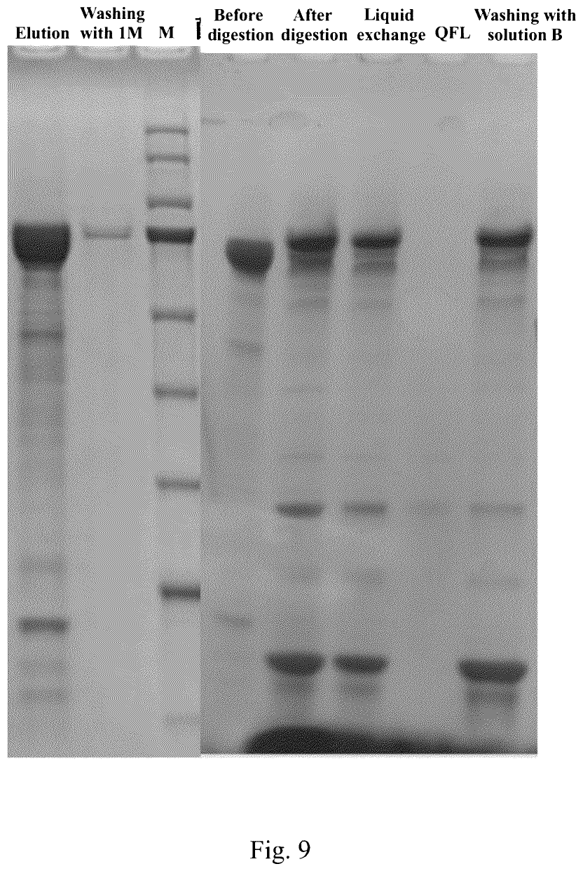

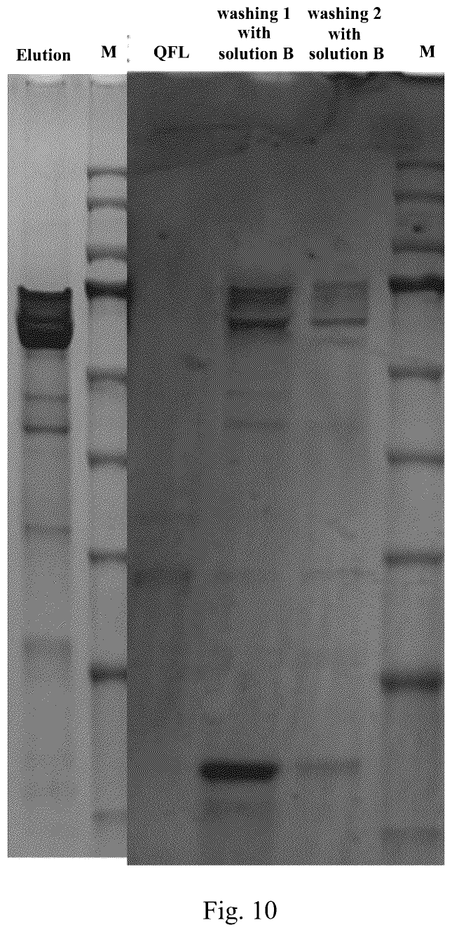

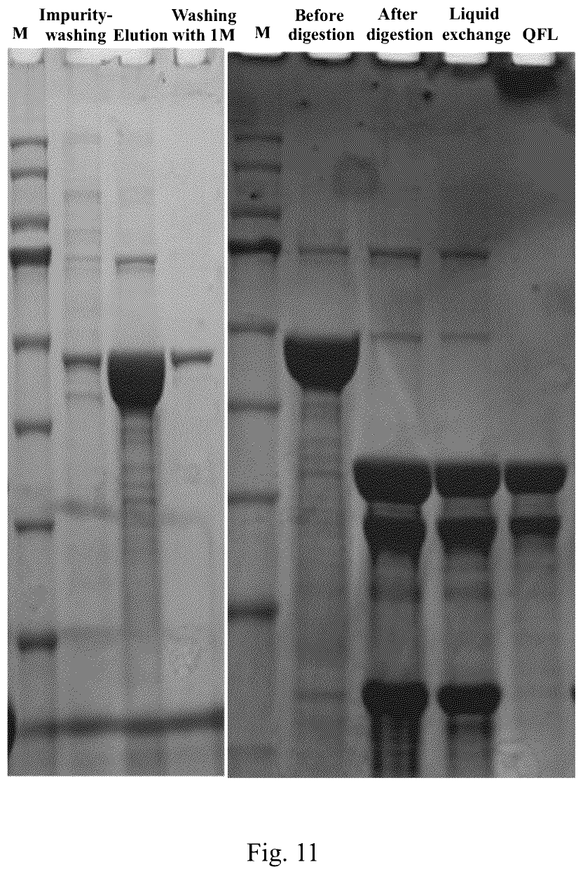

FIG. 1 shows the results of electrophoresis detection of C4P7Ca. FIG. 2 shows the results of electrophoresis detection of C4P7Cb. FIG. 3 shows the results of electrophoresis detection of C4P7Cc. FIG. 4 shows the results of electrophoresis detection of C4P7Cd. FIG. 5 shows the results of electrophoresis detection of C4P7Ce. FIG. 6 shows the results of electrophoresis detection of C4P7Cf. FIG. 7 shows the results of electrophoresis detection of C4P7Cg. FIG. 8 shows the results of electrophoresis detection of C4P7Ch. FIG. 9 shows the results of electrophoresis detection of C4P7Ea. FIG. 10 shows the results of electrophoresis detection of C4P7Eb. FIG. 11 shows the results of electrophoresis detection of C4P7Ec. FIGS. 1-11 show that the actual molecular weights of the isolated proteins (C4P7Ca, C4P7Cb, C4P7Cc, C4P7Cd, C4P7Ce, C4P7Cf, C4P7Cg, C4P7Ch, C4P7Ea, C4P7Eb, and C4P7Ec) are consistent with their corresponding expected molecular weights, demonstrating that the proteins are correctly expressed.

### Example 2: Mass Spectrometric Detection of Recombinant Type IV Humanized Collagens

TABLE 1
Experimental method

| Instrument | Matrix-assisted laser desorption | | |
|---|---|---|---|
| Name | ionization-time of flight mass spectrometer | | |
| | MALDI-TOF/TOF UltraflextremeTM , Brucker, Germany | | |
| Matrix | CHCA | Laser energy | 125 |
| Data | Mascot | Retrieval species | All species |
| retrieval | | | |
| software | | | |

| | |
|---|---|
| Retrieval | Provided sequences as library |
| database | |

The protein samples (collagens C4P7Cf and C4P7Ch) were reduced with DTT and alkylated with iodoacetamide, followed by the addition of trypsin for enzymatic digestion overnight. The peptide fragments obtained after enzyme digestion were desalted using C18ZipTip, mixed with the matrix a-cyano-4-hydroxycinnamic acid (CHCA), and spotted onto a plate. Finally, the matrix-assisted laser desorption ionization-time of flight mass spectrometer (MALDI-TOF/TOF Ulraflextreme™, Bruker, Germany) was used for analysis (see Protein J. 2016;35:212-7 for peptide fingerprinting technology).

Data retrieval was performed using the MS/MS lon Search page from the local Mascot website. The protein identification results were obtained based on the primary mass spectrometry of the peptide fragments generated after enzymatic digestion. Detection parameters: trypsin enzymatic digestion, allowing two missed cleavage sites. The alkylation of cysteine was set as a fixed modification, and the oxidation of methionine was set as a variable modification. The database used for identification was NCB prot.

TABLE 2
Molecular weights and corresponding polypeptides detected by mass
spectrometry of recombinant type IV humanized collagen C4P7Cf

| Start- | Observed | Mr (expected | |
|---|---|---|---|
| End | value | value) | Peptide |
| 241-276 | 3353.6817 | 3352.6744 | GSKGEVGFPGLAGSPGIPGSKGEQGFM |
| | | | GPPGPQGAK (SEQ ID NO: 39) |
| 244-276 | 3065.6004 | 3064.5932 | GEVGFPGLAGSPGIPGSKGEQGFMGPP |
| | | | GPQGAK (SEQ ID NO: 40) |
| 244-279 | 3381.6741 | 3380.6668 | GEVGFPGLAGSPGIPGSKGEQGFM GPP |
| | | | GPQGARGDK (SEQ ID NO: 41) |
| 262-276 | 1457.6553 | 1456.6480 | GEQGFMGPPGPQGAK (SEQ ID NO: 42) |
| 262-276 | 1473.6731 | 1472.6658 | GEQGFMGPPGPQGAK (SEQ ID NO: 42) |
| 262-279 | 1773.7801 | 1772.7728 | GEQGFMGPPGPQGAKGDK (SEQ ID |
| | | | NO: 43) |
| 277-300 | 2199.0695 | 2198.0622 | GDKGSKGEVGFPGLAGSPGIPGSK |
| | | | (SEQ ID NO: 44) |
| 280-300 | 1898.9500 | 1897.9427 | GSKGEVGFPGLAGSPGIPGSK (SEQ ID |
| | | | NO: 45) |
| 280-312 | 3061.5039 | 3080.4966 | GSKGEVGFPGLAGSPGIPGSKGEQGFM |
| | | | GPPGPQ (SEQ ID NO: 46) |
| 280-312 | 3097.4825 | 3096.4752 | GSKGEVGFPGLAGSPGIPGSKGEQGFM |
| | | | GPPGPQ (SEQ ID NO: 46) |
| 283-300 | 1626.8268 | 1625.8196 | GEVGFPGLAGSPGIPGSK (SEQ ID NO: |
| | | | 47) |
| 283-312 | 2825.2583 | 2824.2510 | GEVGFPGLAGSPGIPGSKGEQGFMGPP |
| | | | GPQ (SEQ ID NO: 48) |
| 301-312 | 1217.5133 | 1216.5060 | GEQGFM GPPGPQ (SEQ ID NO: 49) |

The coverage rate of the detected polypeptide segments was 99.04% compared with the theoretical sequence

(GAKGDKGSKGEVGFPGLAGSPGIPGSKGEQGFMGPPGPQGAKGD
KGSKGEVGFPGLAGSPGIPGSKGEQGFMGPPGPQGAKGDKGSKGE
VGFPGLAGSPGIPGSKGEQGFMGPPGPQGAKGDKGSKGEVGFPGL
AGSPGIPGSKGEQGFMGPPGPQGAKGDKGSKGEVGFPGLAGSPGI
PGSKGEQGFMGPPGPQGAKGDKGSKGEVGFPGLAGSPGIPGSKGE
QGFMGPPGPQGAKGDKGSKGEVGFPGLAGSPGIPGSKGEQGFMGP
PGPQGAKGDKGSKGEVGFPGLAGSPGIPGSKGEQGFMGPPGPQ,

SEQ ID NO: 5, in which the covered part is underlined), thus the detection results were very credible.

TABLE 3
spectrometry of recombinant type IV humanized collagen C4P7Ch

| | | Mr | |
|---|---|---|---|
| Start- | Observed | (expected | |
| End | value | value) | Peptide |
| 247- | 2469.2114 | 2468.2041 | GSKGEVGFPGLAGSPGIPGSKGEQGAK (SEQ ID |
| 273 | | | NO: 50) |
| 250- | 2197.0655 | 2196.0782 | GEVGFPGLAGSPGIPGSKGEQGAK (SEQ ID NO: |
| 273 | | | 51) |
| 250- | 2497.1996 | 24961923 | GEVGFPGLAGSPGIPGSKGEQGAKGDK (SEQ ID |
| 276 | | | NO: 52) |
| 277- | 1898.9493 | 1897.9421 | GSKGEVGFPGLAGSPGIPGSK (SEQ ID NO: 53) |
| 297 | | | |
| 280- | 1626.8349 | 1625.8276 | GEVGFPGLAGSPGIPGSK (SEQ ID NO: 54) |
| 297 | | | |
| 280- | 1940.9073 | 1939.9000 | GEVGFPGLAGSPGIPGSKGEQ (SEQ ID NO: 55) |
| 300 | | | |

The coverage rate of the detected polypeptide segments was 98% compared with the theoretical sequence

(GAKGDKGSKGEVGFPGLAGSPGIPGSKGEQGAKGDKGSKGEVGF
PGLAGSPGIPGSKGEQGAKGDKGSKGEVGFPGLAGSPGIPGSKGE
QGAKGDKGSKGEVGFPGLAGSPGIPGSKGEQGAKGDKGSKGEVGF
PGLAGSPGIPGSKGEQGAKGDKGSKGEVGFPGLAGSPGIPGSKGE
QGAKGDKGSKGEVGFPGLAGSPGIPGSKGEQGAKGDKGSKGEVGF
PGLAGSPGIPGSKGEQGAKGDKGSKGEVGFPGLAGSPGIPGSKGE
QGAKGDKGSKGEVGFPGLAGSPGIPGSKGEQ,

SEQ ID NO: 2, in which the covered part is underlined), thus the detection results were very credible.

### Example 3: Detection of Bioactivity of Recombinant Type IV Humanized Collagen

The methods for detecting collagen activity can be referred to in Juming Y ao, Satoshi Y anagisawa, Tetsuo A sakura, Design, Expression and Characterization of Collagen-Like Proteins Based on the Cell Adhesive and Crosslinking Sequences Derived from Native Collagens, J Biochem. 136, 643-649 (2004). The specific methods were performed as follows:

(1) The ultraviolet absorption method was used to detect the concentration of protein samples to be detected, including bovine type I collagen (National Institutes for Food and Drug Control, No. 380002), recombinant humanized collagens C4P7Cf and C4P7Ch provided by the present disclosure.

Specifically, the ultraviolet absorption of the samples at 215 nm and 225 nm was measured respectively, and the protein concentrations were calculated using the empirical formula C (μg/mL)=144×(A 215-A 225). Note that the detection should be performed when A 215<1.5. The principle of this method is that the characteristic absorption of peptide bonds under far-ultraviolet light is detected, which is not affected by the chromophore content, with few interfering substances. The method is easy to operate, so it is suitable for the detection of human collagen and analogs thereof that do not develop color with Coomassie brilliant blue. (Refer to the reference Walker J M. The Protein Protocols Handbook, second edition. Humana Press. 43-45). A fter detecting the protein concentration, the concentration of all proteins to be tested was adjusted to 0.5 mg/ml with PBS.

(2) Sample preparation: the sample stock solution was used directly to conduct the experiment. The positive control bovine type I collagen (PC) was diluted to 1 mg/ml with D-PBS for future use; and the negative control was D-PBS buffer (NC).

(3) Coating: different concentrations of collagen (C4P7Cf or C4P7Ch) and the positive control and the negative control were added to an ELISA plate in a volume of 100 μL per well, and 5 replicate wells were set in each group, then incubated at 4° C. overnight.

(4) Blocking: The supernatant was discarded, and 100 μL of 1% BSA (heat-inactivated at 56°° C. for 30 minutes) was added, followed by incubation at 37° C. for 60 minutes. The supernatant was discarded, and the plate was washed 3 times with D-PBS solution.

(5) Cell seeding: 105 well-cultured 3T3/NIH cells resuspended in D-PBS were added to each well and incubated at 37°° C. for 120 minutes. Each well was washed 3 times with D-PBS solution.

(6) Detection: the absorbance at OD 450 nm was detected using CCK 8 detection kit (manufacturer Beyotime Biotech Inc., product catalog number C0038). The adherence degree of the cells was calculated according to the following formula. The adherence rate of cells can reflect the cell-adhesion ability of collagen. The higher the cell adhesion ability, the better the external environment can be provided to the cells in a short time to facilitate cell adherence.

$$p = ( {OD}_{1} - {OD}_{0} ) / ( {OD}_{2} - {OD}_{0} )$$

where:

P: relative cell adhesion ratio;

OD₁: the average ultraviolet absorbance at 450 nm of all replicate wells of the tested collagen sample;

OD₂: A verage ultraviolet absorbance at 450 nm for all replicate wells of the control collagen sample;

OD₀: Average ultraviolet absorbance at 450 nm for all replicate wells of the blank control group.

(7) Statistical analysis: The statistical difference between the target recombinant humanized collagen and the negative control was statistically analyzed by two-tailed t-test, where *, P<0.05; **, P<0.01; ***, P<0.001.

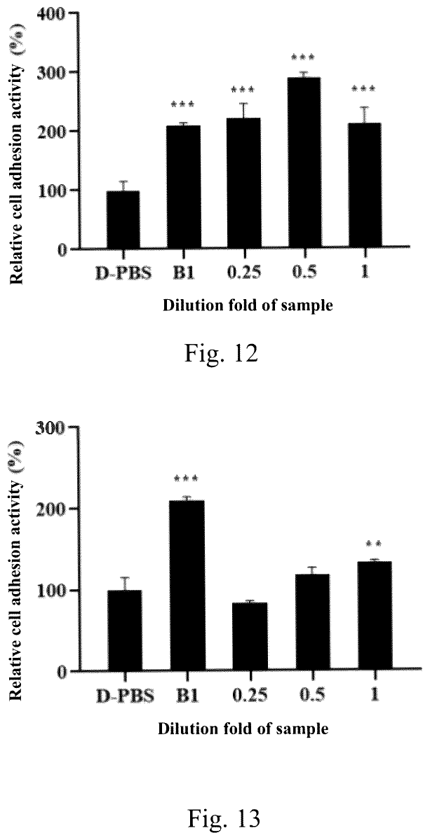

The results were shown in FIGS. 11 and 12. As compared with the D-PBS group, the positive control had a significant effect in promoting cell adhesion, and the recombinant humanized collagen C4P7Cf and C4P7Ch also promote cell adhesion.

Although the present disclosure has been described with reference to illustrative embodiments, those skilled in the art will appreciate that various other changes, omissions and/or additions may be made and that elements of the described embodiments may be replaced with substantial equivalents without departing from the spirit and scope of the disclosure. In addition, many modifications can be made to adapt particular situations or materials to the teachings of the present disclosure without departing from the scope of the present disclosure. Accordingly, it is not intended herein to limit the disclosure to the specific embodiments disclosed for carrying out the disclosure, but rather it is intended that the disclosure will include all embodiments falling within the scope of the appended claims.

1. A recombinant collagen comprising one or more repeating units linked directly or via a linker, wherein the repeating units comprise an amino acid sequence selected from the group consisting of SEQ ID NO: 1 or 28, or a variant thereof, and wherein the variant is (1) an amino acid sequence having one or more amino acid residue mutations in said amino acid sequence of SEQ ID NO: 1 or 28 or (2) an amino acid sequence having at least 80%, 85%, 86%, 87%, 88%, 89%, 90%, 91%, 92%, 93%, 94%, 95%, 96%, 97%, 98%, 99% or 100% identity to said amino acid sequence of SEQ ID NO: 1 or 28.

2. The recombinant collagen according to claim 1, comprising an amino acid sequence selected from the group consisting of SEQ ID NO: 2, 5, 8, 11, 14, 17, 20, 23, 26, 29 or 32, or a variant thereof, wherein the variant is (1) an amino acid sequence having one or more amino acid residue mutations in said amino acid sequence or (2) an amino acid sequence having at least 80%, 85%, 86%, 87%, 88%, 89%, 90%, 91%, 92%, 93%, 94%, 95%, 96%, 97%, 98%, 99% or 100% identity to said amino acid sequence of SEQ ID NO: 2, 5, 8, 11, 14, 17, 20, 23, 26, 29 or 32.

3. A nucleic acid encoding the recombinant collagen according to claim 1.

4. A vector comprising the nucleic acid according to claim 3.

5. A host cell comprising the nucleic acid according to claim 3.

6. A composition comprising the recombinant collagen according to claim 1.

7. A method of producing one or more of a biological dressing, a human bionic material, a plastic surgery or beauty material, an organoid culture material, a cardiovascular stent material, a coating material, a tissue injection filling material, an ophthalmic material, an obstetrics and gynecology biomaterial, a nerve repair and regeneration material, a liver tissue material and blood vessel repair and regeneration material, a 3D printed artificial organ biomaterial, a cosmetic raw material, a pharmaceutical excipient, and a food additive, comprising using the recombinant collagen according to claim 1.

8. A method of facilitating cell adhesion comprising the step of contacting the recombinant collagen according to claim 1.

9. A beauty method comprising administration of the recombinant collagen according to claim 1 to a subject.

10. A method of producing the recombinant collagen according to claim 1, comprising:
(1) incubating a host cell under a suitable culture condition;
(2) harvesting the host cell and/or culture medium comprising the recombinant collagen; and
(3) purifying the recombinant collagen.

11. The recombinant collagen according to claim 1, wherein the repeating units is 2-50 repeating units.

12. The recombinant collagen according to claim 1, wherein the linker comprises one or more amino acid residues.

13. The recombinant collagen according to claim 1, wherein the variant of SEQ ID NO: 1 is selected from the group consisting of SEQ ID NO: 4, 7, 10, 13, 16, 19, 22, or 31; and/or the variant of SEQ ID NO: 28 is SEQ ID NO: 25.

14. The recombinant collagen according to claim 1, wherein the variant of SEQ ID NO: 1 comprises the following mutations: the addition of GFPGFP (SEQ ID NO: 34) or the N-terminal truncated fragment of SEQ ID NO: 34 with a length of 1-5 amino acids at the N-terminus of the amino acid sequence of SEQ ID NO: 1, and/or addition of GFMGPPGPQGQPGLP (SEQ ID NO: 35) or the C-terminal truncated fragment of SEQ ID NO: 35 with a length of 1-14 amino acids at the C-terminus of the amino acid sequence of SEQ ID NO: 1, or the truncation of the amino acid sequence of SEQ ID NO: 1 at C-terminus by 1-5 amino acid residues; or wherein the variant of SEQ ID NO: 28 comprises the following mutations: addition of Glpgtp (SEQ ID NO: 36) or the N-terminal truncated fragment thereof with a length of 1-5 amino acid residues at the N-terminus of the amino acid sequence of SEQ ID NO: 28.

15. The nucleic acid according to claim 3, which comprises a nucleotide sequence selected from the group consisting of SEQ ID NO: 3, 6, 9, 12, 15, 18, 21, 24, 27, 30 or 33.

16. The vector according to claim 4, which comprises an expression control element, nucleotides of a purification tag and/or nucleotides of a leader sequence operably linked to the nucleic acid.

17. The host cell according to claim 5, which is a eukaryotic cell or a prokaryotic cell.

18. The composition according to claim 6, which is one or more of a biological dressing, a human bionic material, a plastic surgery or beauty material, an organoid culture material, a cardiovascular stent material, a coating material, a tissue injection filling material, an ophthalmic material, an obstetrics and gynecology biomaterial, a nerve repair and regeneration material, a liver tissue material and blood vessel repair and regeneration material, a 3D printed artificial organ biomaterial, a cosmetic raw material, a pharmaceutical excipient, and a food additive.

19. The composition according to claim 6, which is in the form of a solution, a lyophilized powder, a gel, a sponge, or a fiber.

20. The method according to claim 9, wherein the administration is a topical administration, oral administration or injection administration.
